(19) United States
(12) Patent Application Publication
(10) Pub. No.: US 2006/0251630 A1
(43) Pub. Date: Nov. 9, 2006

(54) Encapsulated cell therapy

(75) Inventors: Duncan J. Stewart, Ontario (CA); David Courtman, Toronto (CA); Golnaz Karoubi, Toronto (CA)

Correspondence Address: THE WEBB LAW FIRM, P.C., 700 KOPPERS BUILDING, 436 SEVENTH AVENUE, PITTSBURGH, PA, 15219, US

(73) Assignee: AN-GO-GEN INC., ONTARIO (CA)

(21) Appl. No.: 10/540,743

(22) PCT Filed: Dec. 24, 2003
(86) PCT No.: PCT/CA03/02008

§ 371 Date: Apr. 7, 2006

Related U.S. Application Data
(60) Provisional application No. 60/435,858, filed on Dec. 24, 2002.

(51) Int. Cl. A61K 48/00; C12N 5/08

(52) U.S. Cl. 424/93.21; 435/366; 435/455

(57) ABSTRACT

Cell therapy comprises encapsulating a cell with an integrin or other cell interaction factor, for administration to a mammal. The cells may express a therapeutic transgene or the cells may be regenerative.

### CROSS-REFERENCE TO RELATED APPLICATION

This application claims priority from U.S. patent application Ser. No. 60/435,858 filed Dec. 24, 2002 and currently pending. The entire disclosure of that application is incorporated herein by reference.

### FIELD OF THE INVENTION

This invention relates to cell-based therapy in a mammalian patient using encapsulated cells.

### BACKGROUND OF THE INVENTION

Cell therapy involves the administration of cells which have been selected, multiplied and pharmacologically treated or altered (i.e. genetically modified) outside of the body (Bordignon et al, 1999). The aim of cell therapy is to replace, repair or enhance the biological function of damaged tissues or organs (Bordignon et al, 1999). The use of transplanted cells has been investigated for the treatment of numerous endocrine disorders such as anemia and dwarfism, hematological disorders, kidney and liver failure, pituitary and CNS deficiencies and diabetes mellitus (Uludag et al, 2000).

Transplanted cells may function by releasing naturally occurring bioactive compounds such as growth factors, hormones, or neurotransmitters which are absent or produced in insufficient quantities in an effected system. Examples include the implantation of pancreatic islet cells for the treatment of insulin-dependent diabetes mellitus (Miyamoto, 2001) and the implantation of dopamine producing neurons for the treatment of Parkinson's disease (Lindvall and Hagell, 2001). Therapeutic applications for cell therapy have also been suggested in the areas of diabetes and neural degenerative diseases such as Alzheimer's Disease, and epilepsy. Additionally, cells have also been shown to have great therapeutic potential for the removal of detrimental substances from the body. For example, hepatocytes have been implanted for the treatment of high cholesterol levels as shown by Wang et al., Transplantation Proceedings, 23:894-895 (1991). Cell therapy provides several advantageous over the use of more conventional pharmacological treatments including: localized. delivery of the therapeutic, continuous delivery and the ability to adjust production in response to natural feedback mechanisms (Uludag et al, 2000).

Another use for cell therapy is the enhancement of immune responses through the administration of different types of lymphocytes. Adoptive immunotherapy has been shown to be useful for the treatment of certain cancers such as leukemia where infused cells secrete lymphokines which activate tumour specific cytotoxic responses (Bordignon et al, 1999). Immunotherapy involving virus specific T-cells may also be useful for the treatment of persistent viral diseases such as Epstein-Barr virus (Bordignon et al, 1999).

In comparison to whole organ transplants, cell therapies are more easily available. However, rejection of the transplanted cells by the recipient's immune system is still an issue especially where long term use is desired such as in the case of islet implants for diabetic patients (Morris, 1996). As an alternative to immunosuppression, encapsulation methods have been developed whereby the transplanted cells are physically protected from the recipient's immune system by a membrane barrier (Morris, 1996). The use of encapsulated cells is preferable since the systemic administration of immunosuppressant drugs is associated with deleterious side effects and complications due to non-specific suppression of the immune system (Morris, 1996).

Encapsulation methods are generally classified into two categories:

(1) microencapsulation, typically involving small spherical vesicles ranging in size from 0.3 to 1.5 mm in diameter containing individual cells or small cell masses and

(2) macroencapsulation, which involve the larger cell masses in tubular or disc shaped hollow devices (Uludag et al, 2000).

It is believed that, ideally, the membrane will protect the encapsulated cells from immune responses while at the same time be sufficiently permeable to allow for the influx of molecules necessary for cell survival and the secretiori of the desired bioactive compounds and waste products. Numerous materials have been employed for cell encapsulation with the polysaccharide alginate being the most common (Rowley et al, 1999). Membranes are typically composed of oppositely charged natural or synthetic polymers which form gelled complexes; with the combination of polyanionic alginate and polycationic poly(L-lysine) being widely used (Uludag et al, 2000). By varying the concentration of the respective polymers and their contact time, porosity of the resultant hydrogel membrane can be modulated (Uludage et al, 2000). Other commonly used materials include (meth)acrylmates which tend to be more toxic and agarose, a neutral polymer (Uludag et al, 2000).

Cells or cell masses may be encapsulated by conformal coating techniques whereby the membrane is in direct contact with the cells (Uludag et al, 2000). Alternatively, the membrane may be formed around a core containing the cell mass. The core may be engineered to include components which promote cell survival or cell function such as the inclusion of nutrients and trophic factors.

Membranes or cores may also be engineered to function as a synthetic extracellular matrix (ECM). The addition of ECM components may assist cells in the expression of differentiated functions and the organization of the cell mass within the capsule (Uludag et al, 2000). The use of synthetic ECM has been investigated in relation to adherent cells since the hydrophilic nature of most alginate membranes generally excludes the cell attachment and spreading (Rowley et al, 1999).

Alginate hydrogel sheets covalently modified with RGD-containing ligand have been shown to support the growth of myoblasts (Rowley et al, 1999). Cell interaction with modified alginate hydrogels have only been achieved where the cells are grown on flat sheets, as opposed to enclosed capsules (Rowley et al, 1999).

Thus, in vitro, the prior art has focused on the use of encapsulation techniques increasing the durability of cells and stabilizing the cell environment for increased cell survival. In vivo, the prior art has focused on encapsulation as a means to reduce the recipient's immune response in order to promote cell survival. The prior art is deficient in encapsulation methods which allow for the interaction between encapsulated cells and their capsule. Further, the prior art is deficient in encapsulation methods which allow the encapsulated cells to interact with specific molecules exterior to their capsules. The prior art is also deficient in encapsulation methods which allow encapsulated cells to selectively shed their capsule.

### SUMMARY OF THE INVENTION

It is an object of the present invention to provide novel procedures of cell therapy using encapsulated mammalian cells.

It is a further and more specific object of the invention to provide an encapsulation medium containing biological factors capable of interacting with the encapsulated cells which improve cell survival in vivo or which control a desired differentiation state.

It is a further and more specific object of the invention to provide novel procedures of cell-based therapy whereby encapsulated cells can interact with specific molecules exterior to the capsule through biological factors contained in the encapsulation medium, which factors promote specific cell contact and adhesion.

It is a further and more specific object of the invention to provide novel encapsulation medium capable of promoting or improving the transfer of genes, proteins, or factors into the encapsulated cells.

The present invention teaches a method of preparing a cell comprising encapsulating the cell in a cell encapsulation medium in vitro to form an encapsulation product for use in cell therapy in vivo wherein the encapsulation product includes an integrin binding partner.

In various embodiments, the integrin binding partner is selected from the group consisting of collagen, Fibronectin, Fibrinogen, laminin, thrombospondin, vitronectin, factor X, C3bi, Ig-like cell adhesion molecule (ICAM-1,2,3), type 1 collagen, vascular cell adhesion molecule (VCAM-1), mucosal addressin cell adhesion molecule-1 (MAdCAM-1), vitronectin, coilagens, laminin, LFA, Mac-1, tenascin, von Willebrand factor, thrombospondin, factor X, FXIII, FXIIIa, Arg-Gly-Asp, Leu-Asp-Val, His-His-Leu-Gly-Gly-Ala-Lys-Gln-Ala-Gly-Asp-Val, an integrin binding partner containing the sequence Arg-Gly-Asp, Leu-Asp-Val, and an integrin binding partner containing the sequence His-His-Leu-Gly-Gly-Ala-Lys-Gln-Ala-Gly-Asp-Val. In a further embodiment, the encapsulation product include FXIIIa, a transglutaminase cross linking agent.

In another embodiment, the encapsulation product may have factors which bind with or otherwise interact with or effect a particular tissue or cell in the host. Examples of such factors include DCAM, ICAM and VCAM.

The integrin binding partner may be bound to the cell. The integrin binding partner may be bound to the cell prior to encapsulation.

In another embodiment, the integrin binding partner is not bound to the cell.

In another embodiment, the integrin binding partner is in the cell encapsulation medium.

In another embodiment, the integrin binding partner is at the surface of the cell encapsulation medium.

In an embodiment, the cell encapsulation medium is alginate, agarose, a natural polymer compatible with the survival and function. of the cell, or a synthetic polymer compatible with the survival and function of the cell.

In another embodiment, the encapsulation product contains one cell.

The invention also teaches a method of preparing a cell for use in vivo comprising encapsulating the cell in a cell encapsulation medium in vitro to form an encapsulation product, wherein the encapsulation product includes an integrin binding partner, and wherein the encapsulation product contains one cell. P= The invention also teaches a method of preparing a cell for storage or transportation for later use in vivo comprising encapsulating the cell in a cell encapsulation medium in vitro to form an encapsulation product, wherein the encapsulation product includes an integrin binding partner.

In an embodiment, the cell encapsulation medium contains a transgene. In another embodiment, the cell contains a transgene. The transgene may be incorporated into the cell by including the transgene in the encapsulation medium.

The invention further comprises the use of a prepared cell of the invention for cell therapy by administration to a patient in need thereof. The administration may be intercardiac.

The invention also teaches kits for carrying out the methods of the invention..

### BRIEF DESCRIPTION OF THE DRAWINGS

FIG. 1 is a pictograph illustrating cell viability by flow cytometry.

FIG. 2 is a graph showing the percentage of viable cells with and without encapsulation grown on untreated plates and polyHEMA coated plates.

FIG. 3 is a pictograph showing forward and side scatter of light encapsulated rat fibroblasts on a flow cytometer.

FIG. 4 is a graph showing ELISA VEGF protein secretion results for transfected cells.

FIG. 5 is a graph showing VEGF secretion from encapsulated and non-encapsulated cells.

FIG. 6 is a graph showing VEGF secretion from encapsulated and non-encapsulated cells.

FIG. 7 is a photograph showing the morphology of encapsulated rat fibroblasts.

FIG. 8 is a graph showing the viability of rat fibroblasts with and without encapsulation.

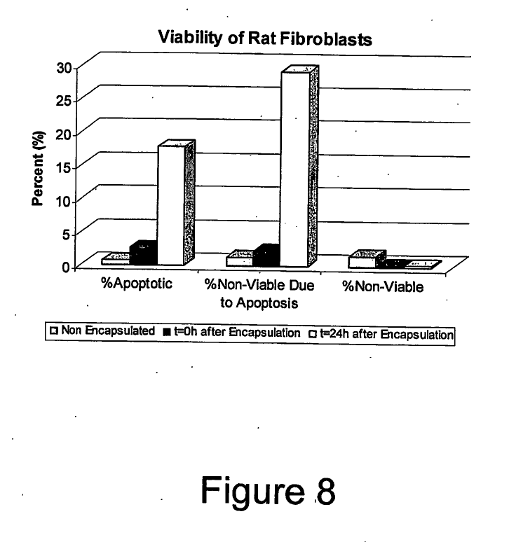

FIG. 9 is a graph showing the viability of encapsulated cells when the encapsulation medium is with or without various integrin binding partners.

FIG. 10 is a photograph shows the morphology of encapsulated cells with or without various integrin binding partners. The top row shows encapsulated cells 30 minutes after plating at 10× magnification (top left image) and at 20× magnification (top right image). The bottom row shows cells encapsulated with agarose and fibronectin, fibrinogen, and vitronectin 30 minutes after plating at 10× magnification (bottom left image) and at 20× magnification (bottom right image).

FIG. 11 is a graph showing the percentage of adherent cells when the encapsulation medium is with or without various integrin binding partners.

FIG. 12 is a graph showing WST-1 assay results for cells where the encapsulation medium is with or without various integrin binding partners.

FIG. 13 is a graph showing the percentage of non-viable cells, with and without encapsulation and with various supplements in the encapsulation medium,

FIG. 14 is a graph showing the number of cells coming out of encapsulation with and without various supplements.

FIG. 15 is a graph showing the percentage of apoptotic and necrotic cells when various levels of integrin binding partners are added to the culture medium.

FIG. 16 is a graph showing the percentage of viable cells without encapsulation, and with different levels of integrin binding partners added to the culture medium.

FIG. 17 is a graph showing the effect of FXIII on cell survival.

FIG. 18 is a graph showing the effect of FXIII on cell proliferation.

FIG. 19 is a graph showing the percentage of viable bone marrow stromal cells when the encapsulation medium is with or without various integrins,

FIG. 20 is a confocal image of CMTMR labelled bone marrow stromal cells (left image) and encapsulate bone marrow stromal cells (right image) injected into the lung microvasculature at 10× magnification.

FIG. 21 is a confocal image of 200 μm lung sections showing engraftment of CMTMR labeled bone marrow stromal cells. The top panel shoes the engraftment of encapsulated bone marrow stromal cells at 1 day after cell injection at 20× magnification (top left image) and 40× magnification (top right image). The bottom panel shows the engraftment of encapsulated bone marrow stromal cells at 7 days after cell injection at 60× magnification.

FIG. 22 is a graph showing the percentage of viable human fibroblast cells when the encapsulation medium is with or without various integrin binding partners.

FIG. 23 is a graph showing mean adhesion data from rat fibroblasts grown in various mediums.

### DESCRIPTION OF THE PREFERRED EMBODIMENTS

Advances in techniques involving adult stem cells and advances in autologous cell therapies have resulted in immune rejection issues becoming less important in cell therapies. Despite this, in vivo cell survival and in vivo cell engraftment remains poor. As shown in the examples below, upon placement in the recipient, cells, whether encapsulated or not, (a) generally don't remain where they are meant to be; (b) stem cells or precursor cells tend to differentiate into cells they were not meant to differentiate into; and (c) cell apoptosis occurs or the cells otherwise do not survive. These problems have led the present inventor to invent further advances in the art. The present invention provides herein a variety of methods and therapies which use cell encapsulation to increase the efficiency of cell therapies.

Firstly, the present invention teaches the use of various factors, such as integrins and matrix molecules, in a cell capsule to interact with the cell and enhance cell survival and selective control of cell differentiation.

Secondly, the present invention teaches the use of various factors, such as integrins and matrix molecules, in a cell capsule to interact with environment outside of the cell and enhance the binding of the encapsulated cell to a selected tissue or organ in the recipient of the cell therapy.

Thirdly, the present invention teaches the use of the cell capsule to retain various factors, such as proteins, drugs, genetic material, for uptake into the encapsulated cell to assist in cell-based therapy. Such uptake may occur through phagocytosis of a portion of the cell encapsulation medium or through other methods known in the art, such as electroporation or viral methods of gene transfer, or, for small molecules, it may occur via passive diffusion through the cell membrane.

Fourthly, the present invention teaches the use of various factors, such as integrins and matrix molecules, in a cell capsule to interact with the cell to control the differentiation of an encapsulated precursor or stem cell before or upon arrival at a selected tissue or organ in the recipient of the cell therapy. The loss of regenerative cells used in cell therapy is largely attributed to ‘anoikis’—programmed cell death in adherence dependent cells due to the loss of integrin-matrix contacts and Cell survival can be enhanced and maintained by using microencapsulation in agarose to promote specific cell matrix interactions in transplanted cells.

Fifthly, the present invention teaches the selection of a cell capsule to enhance the shedding of the encapsulation material from the encapsulated cell upon arrival at a selected tissue or organ in the recipient of the cell therapy.

Sixthly, the present invention teaches the selection of a cell capsule to provide mechanical advantages which enhance cell survival in vivo and which enhance cell migration. For example, by selection of the diameter of the encapsulated cell, the present invention improves the ability of the cell to lodge at the appropriate organ or tissue (e.g. lodging into the pulmonary microvessels or the kidney tubules). For example, encapsulation will reduce cell shearing for a cell which is to be injected into the body.

The use of genetically modified cells as delivery vehicles in gene therapy is becoming increasingly more significant. And as clinical studies using in vitro engineered cells are approaching, their survival and functionality becomes a central issue in the applicability of these cells. To ensure that the modified cells are viable and functioning to express the therapeutic gene, optimum survival conditions are necessary. In this, micro-encapsulation has the potential in providing optimum survival conditions for the therapeutic cells. The present inventors explored the potential of the agarose micro-capsules in providing a temporary home for individually transfected cells maintaining their viability and functionality. These microcapsules were also applied in transplantation and delivery. The microcapsules are both biodegradable and biocompatible and the agarose matrix can be used for selective targeting in vivo.

The present inventors have developed methods for using micro-encapsulation to investigate the transient gene expression profiles on a single cell level. This provides significant information with regards to transfection efficiency (defined here, as the number of cells expressing the gene of interest), plasmid number per cell and duration of transgene expression.

The present inventors have also employed the encapsulation technique to provide optimum survival conditions for genetically modified cells for use in cell therapy. Thus the present inventors have found methods to obtain longer more stable gene expression as well as to manipulate and engineer better survival conditions for genetically modified cells. The overall goal is to optimize cell based gene therapy.

An enabling technology for the transportation and cell survival of genetically modified cells is thus provided. Both gene expression and cell survival are important in cell based gene therapy. Both of these go towards improving cell based therapy for diseases such as chronic ischemia (heart or limbs), heart failure, and Primary Pulmonary Hypertension and other lung diseases in which cell-based gene therapy offers great potential, such as acute respiratory distress syndrome (disruption of the alveolar-capillary membrane), oxidative lung injury, radiation-induced lung injury and lung inflammation. Common to all these lung diseases is that pulmonary injury may lead to transient or permanent alterations in the structure and function of the epithelium and other lung cell types. A treatment, which would include differentiation into these cell types and regeneration of damaged lung tissue is in most cases be the ideal therapy. The ability to deliver cells with the potential to differentiate into lung cell lineages and if necessary, regenerate damaged tissue is a powerful tool. A component in the development of a cell-based therapeutic system is the selection of the cell type as the vehicle for delivery. The low-pressure system and natural filtering function of the pulmonary microvasculature offers a critical advantage in that cells delivered via the pulmonary circulation will easily migrate and lodge into the lung. In fact the present inventors have previously shown prevention and reversal of primary pulmonary hypertension in the rat model using transiently transfected somatic cells expressing nitric oxide synthase and vascular endothelial growth factor. Although rat fibroblasts show a therapeutic effect, the homing and differentiation potential of stem and progenitor cells remains a crucial factor lacking from current cell types used for treatment. Regulating transgene expression and enhancing the survival of the cell used as the delivery vehicle gives tools to obtain longer more uniform gene expression in cell based gene therapy treatments.

Cell encapsulation ensures the retention of transplanted cells and, since these cells must commit to a migratory phenotype to exit the capsule, this in turn ensures efficient penetration and engraftment of the organ. Thus, the enhanced retention and engraftment of encapsulated cells represents a unique advantage of the present invention. This applies not only to the delivery of regenerative cells through the vasculature, for example injection into the pulmonary or coronary arteries, but also to the direct injection of cells into the target organ, for example into the myocardium of the heart.

Lim, U.S. Pat. Nos. 4,409,331 and 4,352,883, (both of which are incorporated herein by reference, as are all references cited herein) discloses the use of microencapsulation methods to produce biological materials generated by cells in vitro, wherein the capsules have varying permeabilities depending upon the biological materials of interest being produced. Wu et al, Int. J. Pancreatology, 3:91-100 (1988), disclose the transplantation of insulin-producing, microencapsulated pancreatic islets into diabetic rats. Aebischer et al., Biomaterials, 12:50-55 (1991), disclose the macroencapsulation of dopamine-secreting cells.

The term “cell therapy” refers to a therapy comprising injecting, transplanting or otherwise. placing cells into a mammalian body for therapy. The cells may be autologous, the cells may produce a protein, the cells may be regenerative, the cells may be modified, the cells may be genetically modified, the cells may be somatic cells, precursor cells or stem cells.

The term “alginate” refers to any compound consisting of (1-4) linked beta-D-manuronic acid monomers and x-L-guluronic acid monomers.

The term “encapsulating” refers to the process of coating the exterior of individual cells or groups of cells with an artificial membrane.

The term “encapsulating medium” refers to any compound capable of forming an artificial membrane surrounding individual cells or groups of cells.

Representative examples of microencapsulation devices include, but are not limited to, U.S. Pat. Nos. 5,182,111, 5,283,187, and 5,389,535, all issued to Aebischer et al., U.S. Pat. Nos. 4,487,758, 4,673,566, 4,689,293, 4,806,355, and 4,897,758, each issued to Goosen et al., U.S. Pat. No. 4,803,168, issued to Jarvis, Jr., U.S. Pat. Nos. 4,352,883 and 4,391,909, both issued to Lim, U.S. Pat. No. 4,298,002, issued to Ronel et al., and U.S. Pat. No. 4,353,888, issued to Sefton.

In a macroencapsulation device, larger numbers of cells are enclosed in a chamber of some type. These devices have at least one semi-permeable membrane to allow the necessary flow of fluids while safely retaining the cells. Representative examples of macroencapsulation devices include, but are not limited to, U.S. Pat. No. 5,262,055, issued to Bae et al., U.S. Pat. No. 4,911,717, issued to Gaskill, III, U.S. Pat. No. 4,298,002, issued to Ronel et al., U.S. Pat. No. 5,387,237, issued to Fournier et al., PCT/AU90/00281, filed by Baxter International, Inc., U.S. Pat. No. 5,413,471, issued to Brauker et al., U.S. Pat. No. 5,344,454, issued to Clarke et al., U.S. Pat. No. 5,002,661, issued to Chick et al., and PCT/US94/07190, filed by W.L. Gore & Associates, Inc.

The term “encapsulating product” refers to the end result of'the encapsulating process: an individual cell or a group of cells coated with an artificial membrane.

The term “integrin” refers to a polypeptide belonging to the integin family of cell surface receptors.

Integrins in general and their subunits are described in detail in Ruoslahti and Pierschbacher, Science 238:491 (1987), which is incorporated herein by reference All terminology used herein is intended to conform to the definitions and descriptions provided by this reference. These integrins comprise a family of related cell surface heterodimeric glycoproteins that are involved in mediating cell adhesive interactions. The integrins include, but are not limited to, receptors for Fibronectin, vitronectin, collagens, laminin, tenascin, and the cell surface protein IIb/IIIa that recognizes Fibronectin, Fibrinogen, von Willebrand factor and thrombospondin. The leukocyte adhesion receptors LFA-1, Mac-1 and gp 150/95 are also members of the integrin. family of receptors. Examples of such integrins include αᵥβ1 (Fibronectin receptor), αᵥβ3 (vitronectin receptor) and αᵥβ3 (type I collagen receptor).

Integrins are heterodimeric cell surface receptors that are composed of noncovalently associated α and β subunits. Using molecular biology and protein chemistry, a number of α and β subunits have been identified. The integrin family can be subdivided into classes based on the β subunits, which can be associated with one or more a subunits. The most widely distributed integrins belong to the β1 class, also known as the very late antigens (VLA). The second class of integrins are leukocyte-specific receptors and consist of one of three a subunits (αL, αM, or αX) complexed with the β2 protein. The cytoadhesins αIIbβ3 and αvβ3, constitute a third class of integrins. A fourth class of integrins includes α4β7.

A wide variety of proteins serve as ligands for integrin receptors. In general, the proteins recognised by integrins fall into one of three classes: extracellular matrix proteins, plasma proteins, and cell surface molecules. Extracellular matrix proteins such as collagen, Fibronectin, Fibrinogen, laminin, thrombospondin, and vitronectin bind to a number of integrins. Many of these adhesive proteins also circulate in plasma and bind to activated blood cells. Additional components in plasma that are ligands for integrins include Fibrinogen and factor X. Cell-bound complement C3bi and several transmembrane proteins, such as Ig-like cell adhesion molecule (ICAM-1,2,3) and vascular cell adhesion molecule (VCAM-1), which are members of the Ig superfamily, also serve as cell-surface ligands for some integrins. Mucosal addressin cell adhesion molecule-1 (MAdCAM-1) is another member of the Ig superfamily and is bound by the integrin α4β7.

The target amino acid sequences for many integrins have been identified. For example, the target sequence in α5β1, αIIβ3, and αvβ3, is the Arg-Gly-Asp tripeptide found in proteins such as Fibronectin, Fibrinogen, thrombospondin, type 1 colIlagen, vitronectin and vWF. However, the Arg-Gly-Asp sequence is not the only integrin recognition motif used by adhesive ligands. Another integrin α4β1 binds the variable region (CS1) of Fibronectin via the sequence Leu-Asp-Val and the platelet integrin αIIbβ3 also recognises the sequence His-His-Leu-Gly-Gly-Ala-Lys-Gln-Ala-Gly-Asp-Val at the carboxy-terminus of the gamma chain of Fibrinogen.

The term “integrin binding partner” refers to any polypeptide which interacts with any member of the integrin family of cell surface receptors with a high degree of specificity.

The term “non-immobilized biological factors” refers to any polypeptide which causes or enhances cell to cell interaction, but which is not naturally found immobilized'to a cell surface.

A wide variety of encapsulation mediums can be used in the processes and products of the present invention. Examples include: agarose with fibrin, agrarose with Fibronectin, or a combination of Fibronectin and Fibrinogen. Suitable naturally-derived mediums include plant-derived gums, such as the alkali metal alginates and agarose, and other plant-derived substances, such as cellulose and its derivatives (e.g., methylcellulose). Animal tissue-derived mediums such as gelatin and chitosan are also useful. Alternatively, the core matrix can be made of extracellular matrix (ECM) components, as described by Kleinman et al., U.S. Pat. No. 4,829,000. Suitable synthetic hydrogels include polyvinyl alcohol, block copolymer of ethylene-vinylalcohol, sodium polystyrene sulfonate, vinyl-methyl-tribenzyl ammonium chloride and polyphosphazene (Cohen, S. et al. J. Anal. Chem. Soc., 112, pp. 7832-7833 (1990)).

Cells can be encapsulated in hollow fibers or in microcapsules that are several hundred microns in size. The former has the advantage of higher mechanical stability and retrievability. Microcapsules on the other hand have a higher surface to volume ratio for growth of anchorage-dependent cells and lower mass transfer resistance for nutrients supply and product secretion. To combine the strength of the two approaches, microencapsulated cells can further be macroencapsulated, for instance, in hollow fibers; choice of highly permeable hollow fibers would add little to the overall mass transfer resistance.

Microcapsule formulation is a known technology used by the pharmaceutical industry to manufacture sustained release products. In the area of cell encapsulation, gelation of alginates is the most extensively studied system. Alginate is a glycuranan extracted from brown seaweed algae. Calcium or other multivalent counterions chelates contiguous blocks of alpha-1,4-L-guluronan residues present in the polysaccharide. Cell encapsulation is achieved when alginate solution containing suspended living cells is dropped or extruded into a solution containing calcium ions. The microcapsules formed can further be coated by adsorption of polyions such as polylysine, which can be coated by alginate again. Many cell types, including islets, hepatocytes, PC I12 cells, chondrocytes, and fibroblasts, have been encapsulated by this method.

A wide variety of non-immobilized biological factors can be used in the processes and products of the present invention. Examples include: steroids such as testosterone, androgen, gonadotrophins, oestradiol, and progesterone, and NO releasing molecules such as NO-donor compounds.

A wide variety of trans-genes encoding therapeutic factors can be used in the processes and products of the present invention. Therapeutic factors expressed by the trans-genes and delivered by the circulation of other body organs downstream of the lungs are within the scope of this invention.

The genetic material that is delivered to the target cell using the method of the present invention may be genes, for example, those that encode a variety of proteins including anticancer and antiviral agents. Such genes include those encoding various hormones, growth factors, enzymes, cytokines, receptors, MHC molecules, eNOS (endothelial nitric oxide synthase), iNOS (inducible nitric oxide synthase), nNOS (neuronal nitric oxide synthase), and the like.

The term “genes” includes nucleic acid sequences both exogenous and endogenous to cells into which the virus vector, for example, a pox virus such as swine pox containing the human TNF gene may be introduced. Of particular interest for use as genes for delivery are those genes encoding polypeptides either absent, produced in diminished quantities, or produced in mutant form in individuals suffering from a genetic disease, such as a tumor suppressor gene product such as the retinoblastoma gene product, Wilm's Tumor gene product, adenosine deaminase (ADA) or immunoglobulin. Additionally, it is of interest to use genes encoding polypeptides for secretion from the target cell so as to provide for a systemic effect by the protein encoded by the gene. Specific genes of interest include those encoding TNF, TGF-alpha, TGF-beta, hemoglobin, interleukin-1, interleukin-2, interleukin-3, interleukin4, interleukin-5, interleukin-6, interleukin-7, interleukin-8, interleukin-9, interleukin-10, interleukin-11, interleukin-12 etc., GM-CSF, G-CSF, M-CSF, SDF-1, human growth factor, co-stimulatory factor B7, insulin, factor VIII, factor IX, PDGF, EGF, NGF, IL-ira, EPO, beta-globin and the like, as well as biologically active muteins of these proteins. Genes for insertion into the viral vectors may be from a variety of species; however, preferred species sources for genes of interest are those species into which the viral vector containing the gene of interest is to be inserted. The gene may further encode a product that regulates expression of another gene product or blocks one or more steps in a biological pathway, such as the sepsis pathway. In addition, the gene may encode a toxin fused to a polypeptide, e.g., a receptor ligand, or an antibody that directs the toxin to a target, such as a tumor cell or a virus. Similarly, the gene may encode a therapeutic protein fused to a targeting polypeptide, to deliver a therapeutic effect to a diseased tissue or organ.

The gene may also encode a marker, such as beta-galactosidase, ds-RED, fluorescent proteins such as GFP, CAT, neomycin or methotrexate resistance, whereby the target cells may be selected or detected. The use of such a marker allows the skilled artisan to screen various viral vectors for those that are non-lytic or non-cytopathic in a particular target host cell. For example, the gene encoding beta-galactosidase (lacZ) can be inserted into a viral vector, the modified virus vector is then introduced into the target host cell and the production of beta-galactosidase is measured. Expression of beta-gal provides an indication of viral infectivity and gene expression.

Other examples include those set out in U.S. application Ser. No. 09/404,652, filed Sep. 24, 1999, by the present inventor, which is incorporated herein by reference. Other examples include:

trans-genes expressing hormones, for example growth hormone for treatment of hypopituitary dysfunction, insulin, (thyroid stimulating hormone (TSH) for treatment hypothyroidism following pituitary failure, and other hormones;

trans-genes expressing beneficial lipoproteins such as Apo 1 and other proteins/enzymes participating in lipid metabolism such as lipoprotein lipase;

trans-genes expressing prostacyclin and other vasoactive substances;

trans-genes expressing anti-oxidants and free radical scavengers;

trans-genes expressing soluble cytokine receptors to neutralize actions of damaging levels of immune mediators, for example soluble TNF receptor, or cytokine receptor antagonists, for example IL1ra;

trans-genes expressing soluble adhesion molecules, for example ICAM-1, to interrupt pathological cell adhesion processes such as those which occur in inflammatory diseases;

trans-genes expressing soluble receptors for viruses to inhibit infection of cells, e.g. CD4, CXCR4, CCR5 for HIV;

trans-genes expressing cytokines, for example IL-2, to activate immune responses for combating infections;

the cystic fibrosis gene, as a trans-gene.

Other examples of trans-genes for use in the cell based therapy of the invention include trans-genes encoding for:

elastase inhibitors for use in treating pulmonary vascular disease such as pulmonary hypertension or systemic vascular disease;

bone morphogenetic proteins (BMP) and BMP receptors 1 and 2, endoglin and genes coding for serotonin receptors or uptake mechanisms for the treatment of genetically based pulmonary arterial hypertension

tissue inhibiting metaloproteinases for use in treating atherosclerosis or arterial aneurysms

potassium channels or potassium channel modulators for use in treating pulmonary hypertension

anti-oxidants such as superoxide dismutase for use in treating pulmonary hypertension, ARDS and pulmonary fibrosis

anti-inflammatory factors such as cytokines, IL-10 and IL-4 for use in treating inflammatory vascular disease such as atherosclerosis or arterial aneurysms

Specific examples of useful angiogenic factors for delivery by way of trans-genes in cells, or by way of other routes of the additional aspect of this invention include vascular endothelial growth factor (VEGF) in all of its various known forms, i.e. VEGF165 which is the commonest and is preferred for use herein, VEGF205, VEGF189,VEGF121,VEGFB and VEGFC(collectively referred to herein as VEGF); fibroblast growth factor (FGF, acid and basic), angiopoietin-1 and other angiopoietins, transforming growth factor—(TGF-), and hepatic growth factor (scatter factor) and hypoxia inducible factor (HIF). VEGF is the preferred angiogenic factor, on account of the greater experience with this factor and its level of effective expression in practice. Specific examples of useful vasoactive factors for delivery by way of trans-genes in cells, or by way of other routes of the additional aspect of this invention include nitric oxide synthase (NOS), PGIS, and hemoxygenase. DNA sequences constituting the genes for these factors are known, and they can be prepared by the standard methods of recombinant DNA technologies (for example enzymatic cleavage and recombination of DNA), and introduced into mammalian cells, in expressible form, by standard genetic engineering techniques such as those mentioned above (viral transfection, electroporation, lipofection, use of polycationic proteins, etc).

In one embodiment, cells of the invention can be used for introduction into the patients pulmonary system. In preparing cells for transfection and subsequent introduction into a patient's pulmonary system, it is preferred to start with somatic mammalian cells obtained from the eventual recipient of the cell-based gene transfer treatment of then present invention. A wide variety of different cell types may be used, including fibroblasts, endothelial cells, smooth muscle cells, progenitor cells (e.g. from bone marrow, adipose, or peripheral blood), dermal fibroblasts, EPC (endothelial progenitor cells) or other mesenchymal stem cells, marrow stromal cells (MSC), and epithelial cells, and others. Dermal fibroblasts are simply and readily obtained from the patients exterior skin layers, readied for in vitro culturing by standard techniques. Endothelial cells are harvested from the eventual recipient, e.g. by removal of a saphenous vein and culture of the endothelial cells. Progenitor cells can be obtained from bone marrow biopsies or isolated from the circulating blood, and cultured in vitro. The culture methods are standard culture techniques with special precautions for culturing of human cells with the intent of re-implantation.

In accordance with an embodiment of the present invention, circulating endothelial progenitor cells (EPCs) or dermal fibroblasts from the patient may be used as the cells for gene transfer. Given the fact that the logical choice of cell types for one skilled in the art to make would be a cell type naturally found in the patient's pulmonary system, such as smooth muscle cells, the use of fibroblasts is counter-intuitive. Surprisingly, it has been found that fibroblasts are eminently suitable for this work, exhibiting significant and unexpected advantages over cells such as smooth muscle cells. They turn out to be easier to grow in culture, and easier to transfect with a trans-gene, given the appropriate selection of technique. They yield a higher proportion of transfectants, and a higher degree of expression of the angiogenic factors in vivo, after introduction into the patient's pulmonary system. They contribute very favourably to the repair of the microvasculature. The anticipated greater risk with fibroblasts of possibly causing fibrosis in the pulmonary system, as compared with smooth muscle cells, has not materialized. Circulating EPCs have been found to be particularly suitable for repair of the microvasculature.

The somatic gene transfer in vitro to the recipient cells, i.e. the genetic engineering, is performed by standard and commercially available approaches to achieve gene transfer, as outlined above. Preferably, the methods include electroporation, the use of poly cationic proteins (e.g. SUPERFECT*) or lipofection (e.g. by use of GENEFECTOR), agents available commercially and which enhance gene transfer. In particular, electroporation provides a high degree of transfection and does not require the use of any foreign material. However, other methods besides electroporation, lipofection and polycationic protein use, such as viral methods of gene transfer including adeno and retro viruses, may be employed. These methods and techniques are well known to those skilled in the art, and are readily adapted for use in the process of the present invention. Electroporation is the most preferred technique, for use with dermal fibroblast host cells and EPCs, providing a higher transfection rate without requiring the use of any foreign material. Polycationic proteins is useful for use with smooth muscle cells. In another embodiment, the present inventors have found good transfection may be achieved by incorporating the gene or a plasmid containing the gene of interest into the capsule. As the capsule degrades, the cell uptakes the gene, likely by endocytosis.

The encapsulated cells can be administered directly to the patient, e.g. by direct infusion of the encapsulated cell suspension, into the vasculature intravenously, or by injection directly into the target tissue, for example heart muscle, by percutaneous or surgical administration. They can also be administered to the lungs of a patient by processes of inhalation.

The re-introduction of the genetically engineered cells into the pulmonary circulation can be accomplished by infusion of the cells either into a peripheral vein or a central vein, from where they move with the circulation to the pulmonary system as previously described, and become lodged in the smallest arterioles of the vascular bed of the lungs. Direct injection into the pulmonary circulation can also be adopted, for example through a Swan Ganz catheter. Injection into the right ventricle or right atrium may be carried out using the pacing port of a Swan Ganz catheter. The infusion can be done either in a bolus form i.e. injection of all the cells during a short period of time, or it may be accomplished by a continuous infusion of small numbers of cells over a long period of time, or alternatively by administration of limited size boluses on several occasions over a period of time.

While the transfected cells themselves are largely or completely retained in the pulmonary circulation, and especially in the arterioles of the patient's lungs, the expression products of the trans-genes thereof are not restricted in this manner. They can be expressed and secreted from the transfected cells, and travel through. the normal circulation of the patient to other, downstream body organs where they can exert a therapeutic effect. Thus, while a preferred use of the process of the invention is in the treatment of pulmonary disorders, since the expression products initially contact the patient's pulmonary system, it is not limited to such treatments. The transfectants can contain trans-genes expressing products designed for treatment of other body organs of the patient. Such products expressed in the pulmonary system will target the other, predetermined organs and be delivered thereto by the natural circulation system of the patient.

An amount effective to treat the disorders hereinbefore described depends on the usual factors such as the nature and severity of the disorders being treated and the weight of the mammal. However, a unit dose will normally contain for example 500,000 to 500 million cells, depending on the size of the recipient and the nature of the therapy (i.e. intravenous versus injection directly into the tissue). The amount of therapeutic material incorporated into the encapsulation medium will vary depending on the potency of the particular agent, from 10⁻⁹ to 10 mg, of the encapsulated cell therapeutic, or a pharmaceutically acceptable salt thereof. Unit doses of the encapsulated therapeutic may be administered once only, or with repeated applications, for example, weekly, monthly, or possibly more than once a day, depending on the half life and purpose, for example 2, 3, or 4 times a day, more usually 1 to 3 times a day, such that the. total daily dose is normally in the range of 0.0001 to 1 mg/kg; thus a suitable total daily dose for a 70 kg adult is 0.01 to 50 mg, for example 0.01 to 10 mg or more usually 0.1 to 10 mg.

As is common practice, the compositions will usually be accompanied by written or printed directions for use in the medical treatment concerned.

In other embodiments, the cells of the invention may not contain transgenes. For example, stem cells, precursor cells, progenitor cells from skin, fat, muscle, bone marrow, blood, or liver, endothelial progenitor cells, embryonic stem cells, islet cells, endocrine cells, neural cells, including neurons and glia, epithelial cells, lung cells, cardiac muscle cells, adult cells or cell cultured from such cells can be used for regenerative or replacement therapies.

The cells of the invention can be delivered to the patient by various methods, including those known in the art, and those described in U.S. Pat. Nos. 6,004,295 and 6,482,406 to the present inventors. For example, the cells can be delivered by injection into the arteriole or venous vascular system, for travel and delivery by lodging in any vascular bed, such as the lungs, kidney, or liver. The appropriate site of injection may be the lung or may be an inter-cardiac injection. Alternatively, the cells can be delivered by direct injection into the tissue or by insertion into the tissue by percutaneous or surgical means.

The invention is further described for illustrative purposes, in the following specific, non-limiting Examples.

### EXAMPLE 1

### Behavious and Viability of Encapsulated Cells

Rat fibroblasts were individually encapsulated with agarose. Empty capsules were prepared in varying percent compositions and kept at cell culture conditions for up to 21 days. The capsules remained intact for the most part. Clumping (two or three capsules sticking together) occurred beginning at day 5 and consistently increased. In some cases “blobs” of agarose were forming.

The cell micro-encapsulation technique was optimized for rat fibroblasts and their behaviour in capsules was studied.

Briefly, methodology developed by Weaver et al. was adapted for the selection of antibody producing hybridoma cells. In gel microdrop assays, specific proteins secreted from single cells are captured and quantified. Biotinylated protein specific capture antibodies are bound to the biotin-derivatized gel matrix through a steptavidin. Proteins secreted by the encapsulated cell bind to the capture antibody sites and are subsequently quantified with a fluorescently labeled reporter antibody. Cells are suspended in a Hank's Buffered Salt Solution and added to 4% agarose, which may be biotinylated. The mixture is then added dropwise to an inert oil, dimethylpolysiloxane. This is then immediately vortexed at the highest speed for 1 minute and immediately placed in an ice bath for 10 minutes. The mixture is then centrifuged at 1800 RPM for 10 minutes and the oil phase and aqueous phase are subsequently removed to give a final phase containing encapsulated cells. The encapsulated cells are then washed with Hank's Buffered Salt Solution and filtered through a 70 micron cell strainer.

Encapsulated cells are processed in bulk culture and individually analyzed using FACS and sorted by methods known in the art.

The fluorescence signal from the specific reporter antibody can be quantified, allowing subsequent isolation and recovery of a subpopulation of cells. As the capture antibodies are linked with the biotin-conjugated agarose through an avidin bridge, any secreted molecule for which there is an appropriate antibody pair can be captured and quantified.

It was found that the encapsulation technique did not have any adverse effects on the cells. Most of the cells remain in the capsules but there is a small percentage that, breaks through the agarose gel and adheres to the bottom of the flask. This mechanism could be observed under microscope. Cells break free of the gels by first attaching to the bottom of the flask and then shedding the agarose coating. As has been observed under microscope, cells migrate to the edge of the gel, break it open and leave the agarose shell behind. It is hypothesized that the viable, healthy cells are the ones breaking free of the gel. As the rat fibroblast cells are adherent, they don't remain viable in the capsules. In fact propidium iodide staining showed that a significant percentage of the cells are not viable after 4 days in culture. Similar observations were made using trypan blue staining and by simply observing the cells under the microscope.

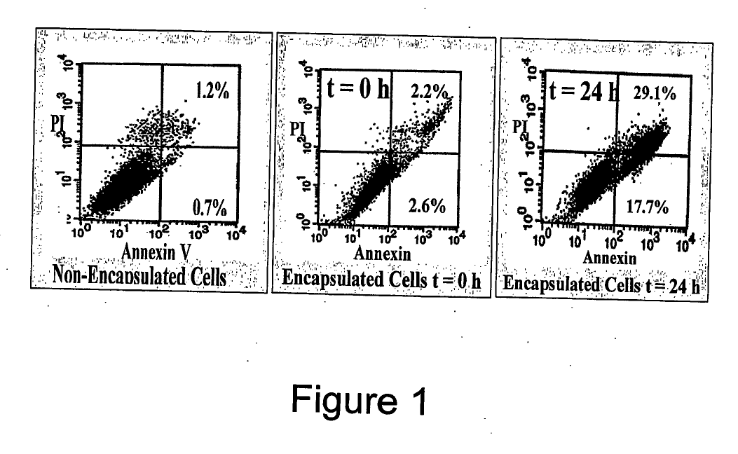

FIG. 1 shows an analysis of cell viability by flow cytometry, using Annexin V intensity (x-axis) and Propidium iodide intensity (y-axis) immediately following encapsulation, and 24 hours later. Rat fibroblasts were encapsulated and stained (O hours or 24 hours) with Annexin V (green fluorescent) which binds to phosphotidylserine on cell surface, an early event in apoptosis. In addition, the cells were also stained with Propidium Iodide (Red fluorescent), which is a nuclear stain. The double stain distinguishes apoptotic cells from those that are necrotic. FIG. 1 illustrates that the encapsulation procedure does not have any immediate (B) detrimental effects when compared to non-encapsulated cells (A). However, after 24 hours (C) in the capsules a considerable percentage of the encapsulated population were apoptotic or dead as a result of apoptosis.

Continued viability of the encapsulated cells was also examined by propidium iodide staining and flow cytometery. The majority of encapsulated cells were found to be viable after 3 days in culture.

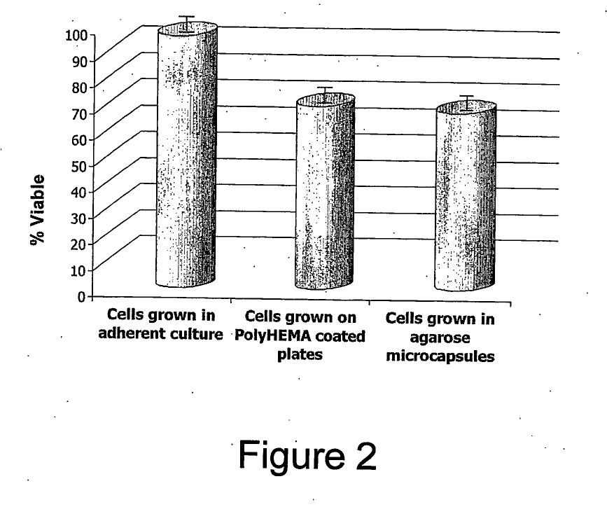

Rat fibroblasts are adherent cells. The viability of encapsulated cells was compared to cells grown in adherent culture and cells grown on PolyHEMA. PolyHEMA is a thin synthetic polymer coating which inhibits the adherence of cells to a tissue culture flask. As shown in FIG. 2, that there is no significant difference between the number of viable cells grown on PolyHEMA coated wells and cells microencapsulated in 2.5% agarose.

To increase the survival of encapsulated cells, Fibrinogen, an ECM component, was incorporated into the agarose gels at concentration of Fibrinogen (0.02 ng/gel) and Fibronectin (0.09 pg/gel). Suitable ranges for integrin concentrations are between 0.01 pg/gel and 0.1 ng/gel, or between 0.05 pg/gel and 0.05 ng/gel, or between 0.09 pg/gel and 0.01 ng/gel. Note that concentrations depend on the potency of the integrin binding partner. For example, Fibrinogen has a more potent effect than Fibronectin or vitronectin on the adhesion and integrin binding properties of the cells. For example, suggested ranges for Fibrinogen are 1.5 to 5 mg per 400 mL of agarose sample. Suggested ranges for Fibronectin are 25 to 200 mg per 400 mL of agarose sample. Suggested ranges for vitronectin are 1.5 to 5 mg per 400 mL of agarose sample. Briefly, to quantify the amount of Fibrinogen remaining on the capsules following the encapsulation process, a GeminiTm spectra max plate reader was used. A standard curve was prepared using the serial dilution to obtain a relationship between the fluorescence intensity of the Oregon green and the concentration of Fibrinogen in the sample. For example, the addition of 1.5 mg of Fibrinogen to the 400 ml (1.87 million microcapsules) sample of agarose resulted in the incorporation of 0.0055 ng/microcapsule.

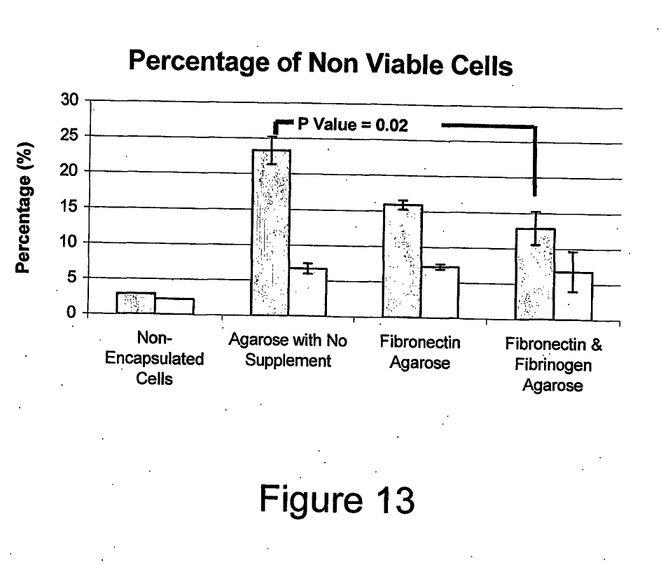

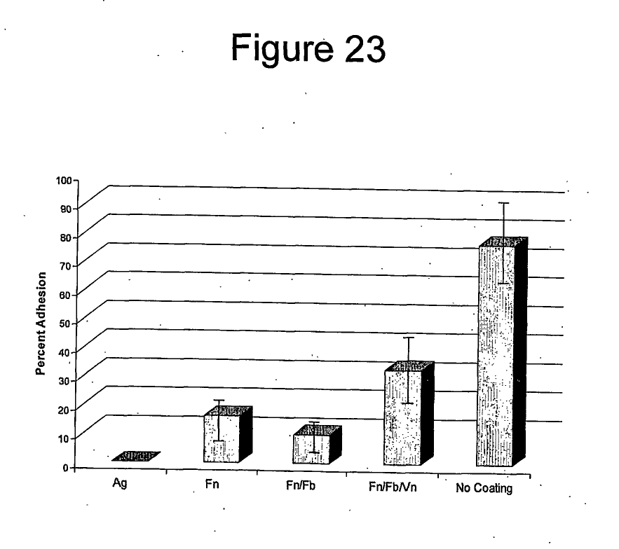

To investigate the mechanism behind this improvement in viability, studies were begun to determine the role of adhesion molecules and integrin binding sites. The effect of fibronectin and fibrinogen in solution were investigated. Results illustrated that the addition of fibronectin and fibrinogen to the culture media of the encapsulated cells (encapsulated in agarose with no supplement), had a significantly detrinental effect on the viability of the cells (FIG. 13). This result is in agreement with anoikis literature in which immobilized ligands induce cell-matrix adhesion through integrins where as soluble ligands have the opposite effect and inhibit the cell matrix adhesions. This further implicates integrin binding as a major factor in the viability of the encapsulated rat fibroblasts. To further assess a role for integrins, adhesion studies comparing the effects of Fibronectin, Fibrinogen and Vitronectin and combinations of the three adhesion molecules have been done. Quantification of the adhesion study implicated a role for of integrins α5β1 and αvβ3 (FIG. 13) (receptors for the above termed ligands) in promoting the adhesion to the supplemented agarose matrix. FIG. 23 shows mean adhesion data from rat fibroblasts grown on a thin coating of 2.5% agarose coated (Ag) or supplemented with fibronectin (Fn), Fibronectin and Fibrinogen (Fn/Fb), Fibronectin & Fibrinogen & Vitronectin (Fn/FbNn) or without any coating. Numbers were obtained by trypsinization and trypan blue staining using a hemocytometer

Cell viability Was examined by light microscopy and cells encapsulated in Fibrinogen capsules appeared healthier as compared to cells in capsules without Fibrinogen. The addition of Fibrinogen was also found to increase the percentage of cells which broke free of the agarose gel and adhered to the flask.

### EXAMPLE 2

### Fibrinogen Improves Cell Viability and Release

To increase the survival of rat fibroblasts in the capsules, Fibrinogen was incorporated into the agarose gels. The overall concentration per gel was found to be 0.0055 ng of Fibrinogen. The addition of Fibrinogen resulted in better survival of the cells. The cells appeared much healthier. Also, the addition of Fibrinogen, it was found that a greater percentage of the cells were breaking out of the capsule and adhering to the flask.

The population characteristics for both the cells and the encapsulated cells were determined using a flow cytometer. The flow cytometer looks at the forward, and side scatter of light. The forward scatter provides information on the size of the cells and the side scatter provides information on the granularity of the cells. The resulting figures illustrate the population profile of the rat fibroblast population and the profile of the encapsulated cell population.

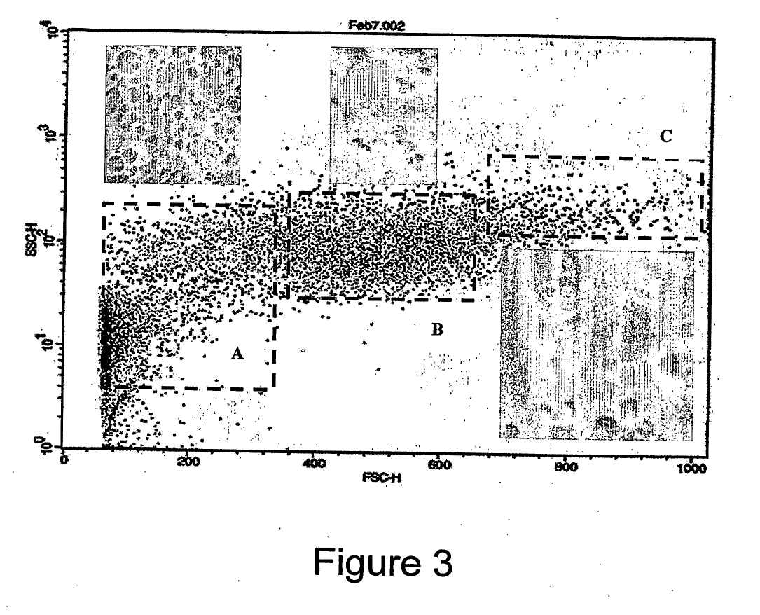

FIG. 3 shows foward and side scatter (representing size and granularity respectively) of light by encapsulated rat fibroblasts as seen on Beckton-Dickinson flow cytometer. Cells were encapsulated in 4% agarose and analyzed by flow cytometry. Selected regions were sorted and analyzed by light microscopy to confirm the profile of the sub-population. The figure shows that 82% of the population is composed of empty agarose beads, while 8.4% of the population are singly encapsulated cells and 6.0% of the population are unusually large agarose beads or multiply encapsulated cells.

### EXAMPLE 3

### Gene Expression Occurs in Encapsulated Cells

Although a significant therapeutic effect is observed with the delivery of transfected cells, appropriate characterization of cell based expression needs to be performed if this therapy is to be optimized. Transient (plasmid based) transfection of cell populations in vitro may result in a wide range of therapeutic protein synthesis when measured in individual cells, and this may likely be the result of varying plasmid copy numbers being introduced per cell. Transfection efficiency, simply measured as the number of cells expressing any detectable level of the transgene, is a primary endpoint measure in all gene therapy experiments. Ideally all cells would contain equal transgene expression, however in practice in vitro transfection efficiencies can be low (even as low as 10-20%, although the present inventors are now achieving 95% transfection efficiency) and the level of gene expression in transfected cells is variable. An understanding of the gene expression profiles on a single cell basis and analysis of expressed protein is important for developing improved cell based therapies.

There are numerous barriers to gene expression each with its own respective regulatory mechanisms. One of the main barriers to gene expression may be the plasmid copy number introduced in each cell during the transfection procedure. It may be that there is an optimum number of plasmid copies that results in gene expression. This example investigates this relationship. Gene expression was investigated in a primary cell line of rat fibroblasts. The cells were transfected by using a plasmid based non-viral technique. This alleviates safety issues surrounding viral transfection methods. The selected gene in this example is VEGF. The transfection results in the secretion of the protein, which will be the end-point measure in the assays. The present inventors developed a cytokine secretion assay for the VEGF transgene expression. This involves using the micro-encapsulation along with an antibody capturing system, which will result in the quantification of the secreted VEGF (on a single cell level) from a transiently transfected rat fibroblast cell line.

In another example, the transfection with eNOS results in the intracellular expression of eNOS protein and synthesis of Nitric Oxide.

Results: VEGF Protein Secretion

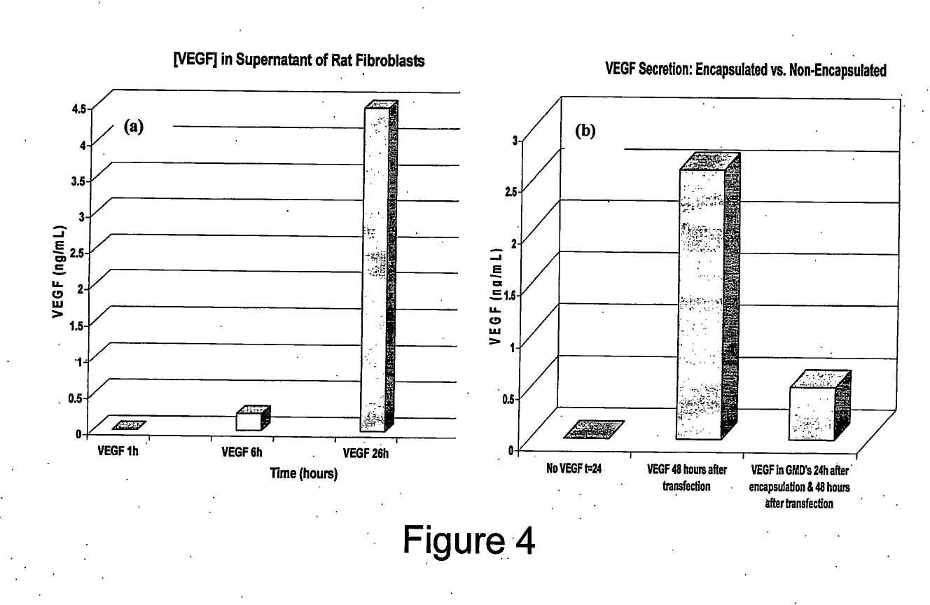

To examine the expression levels and duration of expression, the present inventors carried out several experiments to investigate VEGF expression globally (on a population basis) using ELISA's. FIG. 4 shows ELISA VEGF protein secretion results for transfected cells. The secretion of VEGF from encapsulated cells has also been quantified using the ELISA assay.

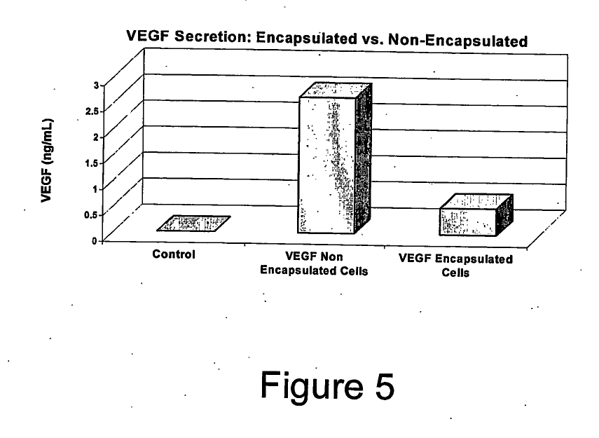

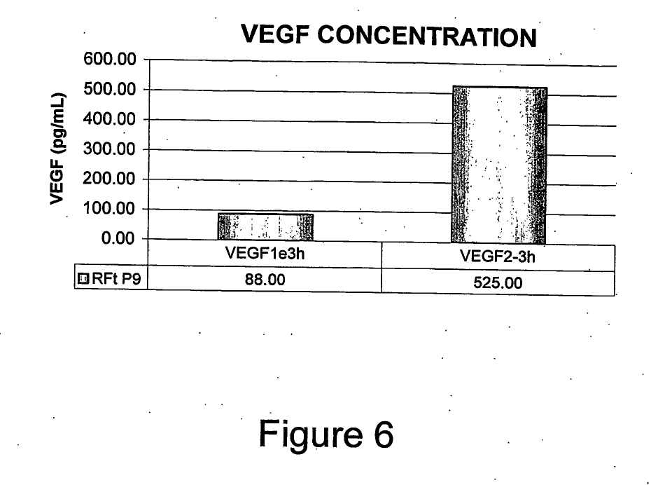

FIG. 5 shows vascular endothelial growth factor (VEGF) secretion from an encapsulated (in 4% Agarose) transfected population of rat fibroblasts, as measured by enzyme linked immuno sorbent assays (ELISA). Rat fibroblasts were transfected with plasmid encoding the VEGF gene. One half of the. population was encapsulated and both subpopulations were incubated for 24 hours to ensure detectable amounts of VEGF. Supernatant was removed from both groups and cells were incubated in fresh media for 3 hours after which samples were taken and analyzed by commercially avalaible ELISA kit. Non-transfected cells were-used as a negative control. FIG. 6 illustrates that although considerably less then the non-encapsulated cells (VEGF2-3h), the encapsulated population (VEGF1e3h) is nevertheless secreting detectable levels of VEGF protein.

Capture Antibody to Protein Ratio.

The appropriate capture antibody to protein ratio was determined to be at least 8:1. Ideally it would be better to use a higher ratio to ensure that the high secreting cells are being captured. The fluorescence intensity was correlated with the amount of VEGF secreted by the encapsulated cells.

### EXAMPLE 4

### Fibronectin and Fibrinogen Factors Promote Survival of Encapsulated Cells

The present inventors investigated the viability of rat fibroblasts in the capsules and the effect of Fibrinogen & Fibronectin on the survival of the encapsulated cells.

Results: Encapsulated Cell Morphology

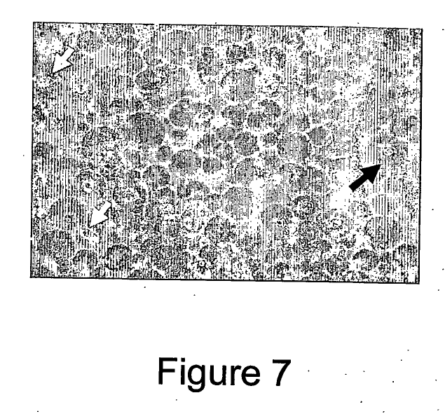

Upon encapsulation cells appear very round and remain that way for varying time periods. Although they are.an adherent population, they do not spread and adhere to the surrounding agarose matrix (FIG. 7). Over time, they appear apoptotic and membrane integrity is lost in many of the cells. As shown in FIG. 7, the Black arrows show the rounded morphology of the cells in the capsule. The white arrow shows the cells that are apoptotic and/or have lost their membrane integrity.

Encapsulated Cell Viability

Using a dual stain for apoptosis and necrosis, the present inventors determined that a small percentage of the cells are apoptotic as a result of the encapsulation process. This number increases to approximately 28% of the cells after 24 hours in the capsules (FIG. 4).

The inventors also found that incorporation of Fibronectin and Fibrinogen into the agarose matrix appears to improve the viability of the cells.

Effect of Fibrinogen & Fibronectin on Encapsulated Cells

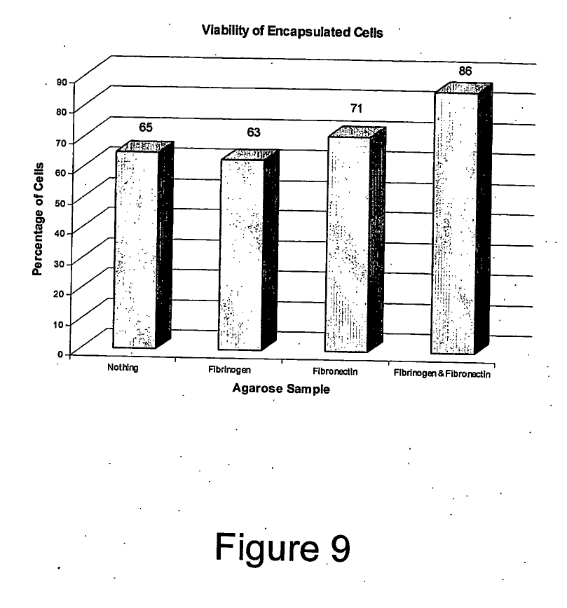

Results indicate that Fibronectin and a combination of Fibronectin and Fibrinogen in the encapsulation medium increase the viability of the rat fibroblasts from 65% to approximately 85% (FIG. 9).

### EXAMPLE 5

### Fibronectin, Fibrinogen and Vitronectin Factors Promote Cell Adhesion

The present inventors investigated the effect of Fibronectin, Fibrinogen, Vitronectin, and combinations of the three adhesion molecules in promoting cell adhesion to a supplemented agarose matrix.

Rat fibroblast were grown on a thin coating of 2.5% agarose (Ag) coated or supplemented with Fibronectin (Fn), Fibronectin and Fibrinogen (Fn/Fb), Fibronectin and Fibrinogen and Vitronectin (Fn/FbNn) or without any coating. Cells were plated at a density of 15000 cells per well in a 24 well plate and allowed to adhere for 1 hour.

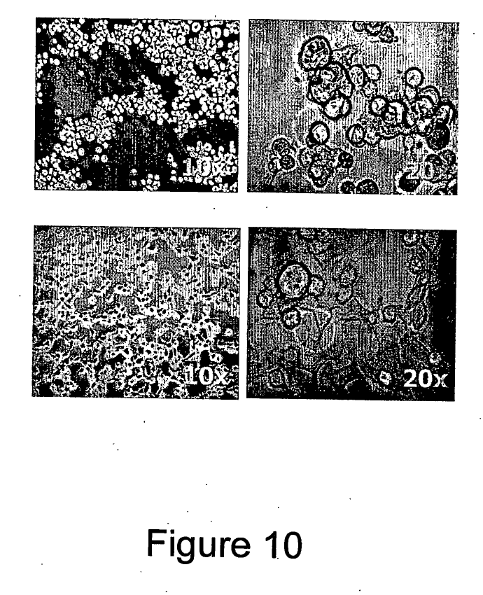

The top panel of FIG. 10 shows the morphology of cells encapsulated in agarose 30 minutes after plating. The bottom panel of FIG. 10 shows the morphology of cells encapsulated in agarose supplemented with Fibronectin and Fibrinogen and Vitanectin 30 minutes after plating.

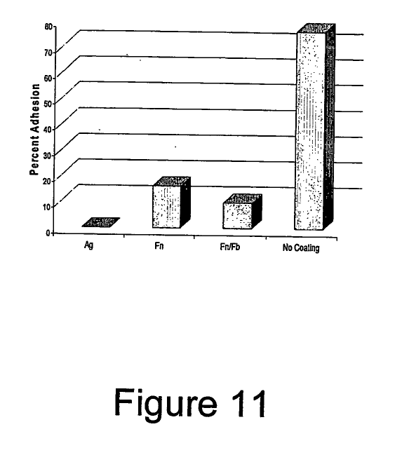

FIG. 11 shows the percentage of adhesive cells as determined by trypsinization and trypan blue staining using a hemocytometer. Results illustrate that the percentage of adherent cells trended upwards with Fibronectin and Fibronectin and Fibrinogen and was significantly increased by the addition of Fibrinogen and Fibronectin and vitronectin into the agarose matrix.

### EXAMPLE 6

### Fibronectin and Fibrinogen Factors Promote Metabolic Activity

The present inventors further investigated the viability of encapsulated rat fibroblasts by examining metabolic activity.

The viability of cells was assessed using the WST-1 assay which assesses the cells ability to convert tertazolium salt (WST-1) to formazon through the enzymatic action of the mitochondrial enzymes. Adherent cells, cells grown on polyHEMA coated wells, cells encapsulated in 2.5% agarose and cells encapsulated in agarose supplemented with Fibronectin and Fibrinogen.

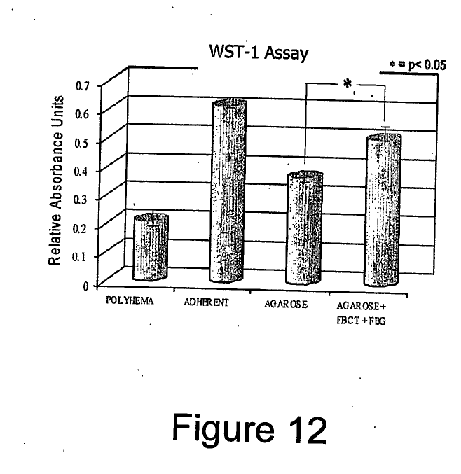

Results indicate that cells encapsulated in supplemented agarose showed increased metabolic activity (FIG. 12).

### EXAMPLE 7

### Fibronectin and Fibrinogen Factors in the Encapsulation Medium Promote Survival and Release of Encapsulated Cells

The present inventors determined the appropriate combination of growth factors and adhesion molecules to improve the viability of the cells in the capsules and to encourage breaking out of the capsules. Other methods of incorporating the growth factors were also investigated. To ensure that the modified cells are viable and functioning to express the therapeutic gene, optimizing survival conditions are important.

Results:

More data was collected on the effect of Fibronectin and Fibrinogen on the viability of the encapsulated rat fibroblasts. FIG. 13 shows mean fluorescence intensity as a result of different supplements in added to the agarose and the effect of agarose supplements on the percentage of apoptotic and necrotic Cells. For each experiment, I million cells were encapsulated. The percentage of apoptotic cells as detected by Annexin V and necrotic cells detected by Propidium Iodide staining. Cells were encapsulated in 4% agarose with no supplement or in 4% agarose gels supplemented with Fibronectin or Fibrinogen & Fibronectin. Results illustrate that the percentage of apoptotic cells trended downwards with Fibronectin and was significantly reduced by the addition of Fibrinogen and Fibronectin into the agarose matrix. Results confirm the initial data showing an improvement in the viability of the cells. Viability results from experiments were statistically significant.

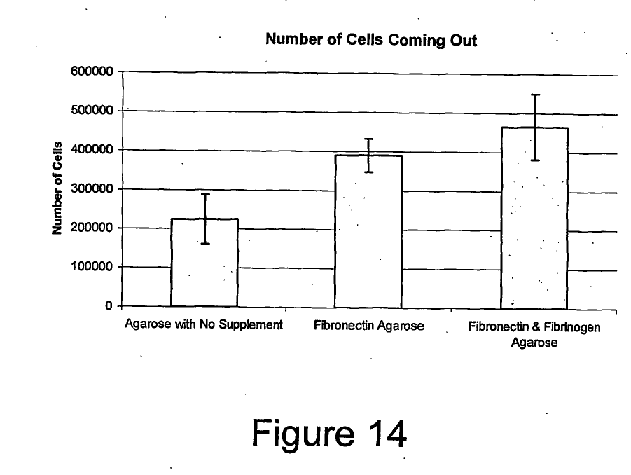

The number of cells coming out of the gels was also significantly improved with the addition of Fibronectin and Fibrinogen (FIG. 14). FIG. 14 compares the number of cells breaking out of the capsule in the no supplement 4% agarose matrix to the 4% agarose matrix supplemented with Fibrinogen and fibronection. The addition of Fibronectin & Fibrinogen to the 4% agarose matrix significantly increased the number of cells breaking out of the capsule and adhering to the bottom of the tissue culture flask (24 hours) as compared to the cells encapsulated with agarose only. This effect was also confirmed by visual (microscopic) observation.

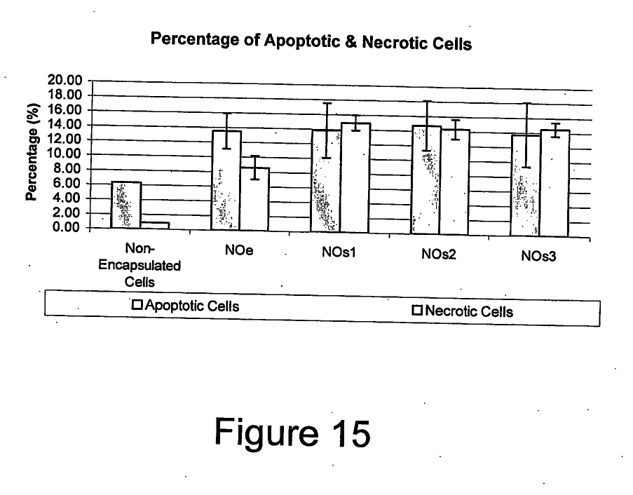

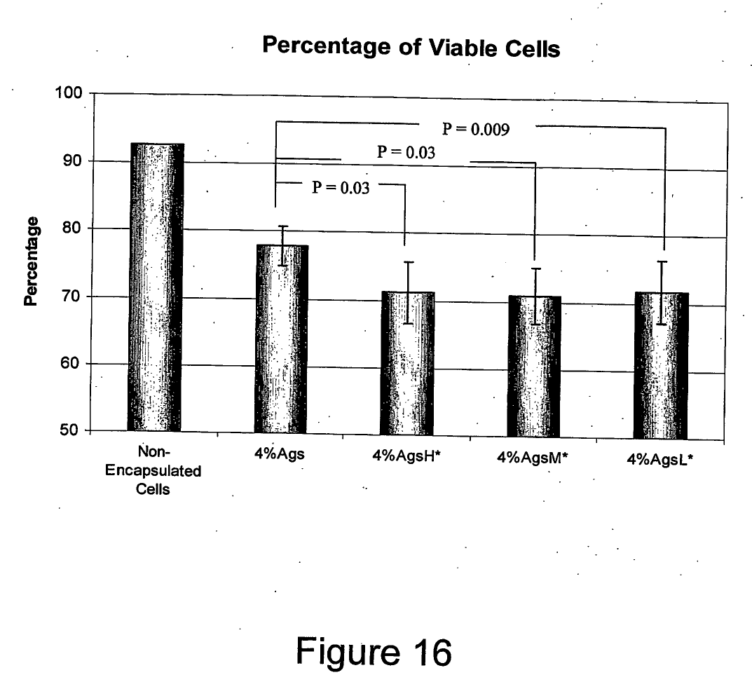

To investigate the mechanism behind this improvement in viability, studies were begun to determine the role of adhesion molecules and integrin binding sites. The present inventors developed a system in which Nitric Oxide production and eNOS protein production can be detected and quantified. Specifically, the effect of Fibronectin and Fibrinogen in the cell culture medium, as opposed to the encapsulation medium was investigated. Results illustrated that the addition of Fibronectin and Fibrinogen to the culture media of the encapsulated cells (encapsulated in agarose with no supplement), had a significantly detrimental effect on the viability of the cells (FIGS. 15 and 16). This further implicates integrin binding via use of an integrin in encapsulation as a major factor in the viability of the encapsulated rat fibroblasts. Studies were also carried out with human fibroblasts. Increased viability is observed in the human fibroblasts encapsulated in the supplemented agarose.

FIG. 15 thus shows the effect of Fibronectin and Fibrinogen added to cell culture media of encapsulated cells. Note that all cells are encapsulated in agarose with no supplement. NOs1, NOs2 and NOs3 represent different concentrations of Fibronectin and Fibrinogen added to cell culture media (0.5 μg Fibronectin+25 μg of Fibrinogen, 2 μg of Fibronectin+100 μg Fibrinogen, 5 μg of Fibronectin+250 μg of Fibrinogen, respectively). In each case 300 000 cells were encapsulated.

FIG. 16 shows the effect of Fibronectin and Fibrinogen added to cell culture media of encapsulated cells and the role of integrin-extracellular matrix protein interactions. In this experiment cells were encapsulated in 4% agarose. Cells were divided into 4 groups and cultured for 24 hours under different culture conditions to investigate the effect of the addition of extracellular proteins to the cells' culture media. The viability results were compared to the controlled group of non-encapsulated cells. The first group contained encapsulated cells incubated for 24 hours in the regular rat fibroblast culture conditions of DMEM +10% FBS +2% PiS (4%Ags). The second, third and fourth groups were encapsulated cells incubated in DMEM +10% FBS +2% P/S supplemented with varying concentrations of soluble Fibronectin and Fibrinogen. Group 4%AgsL* represents the encapsulated cells cultured in 0.5 μg/mL Fibronectin and 25 μg/mL of Fibrinogen. Group 4%AgsM* represents encapsulated cells cultured in 2 μg/mL of Fibronectin and 100 μg/mL of Fibrinogen. And finally, group 4%AgsH* represents encapsulated cells cultured in 5 μg/mL of Fibronectin and 250 μg/mL of Fibrinogen. The addition of Fibronectin and Fibrinogen in solution (i.e. in the cell culture medium, as opposed to the encapsulation medium) appeared to have a detrimental effect on the viability of the cells, although the addition of Fibronectin and Fibrinogen at the chosen concentrations did not have a dose dependent effect. Results were obtained by Annexin V & Propidium Iodide staining. The y-axis represents the percentage of Annexin V and Pi positive encapsulated cells.

### EXAMPLE 8

### FXIII Factor Regulates the Proliferation of Cells

The present inventors investigated the effect of FXIII on the survival of the encapsulated cells.

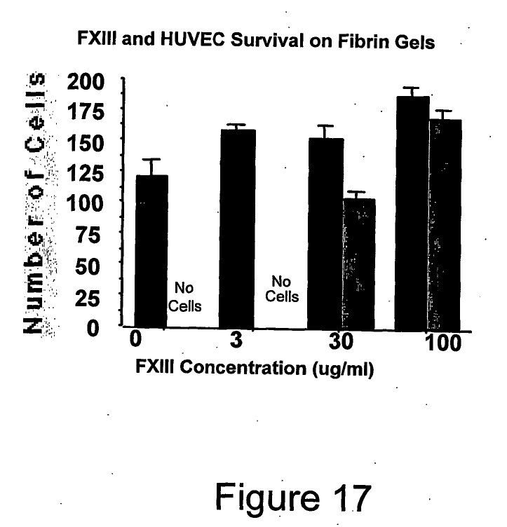

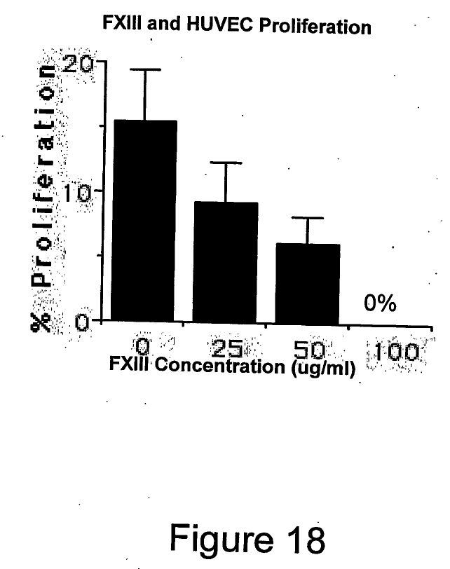

FIG. 17 and 18 show the effect of FXIII cross-linking on Human Umbilical Vein Endothelial Cell (HUVEC) phenotype was examined on thick fibrin gels. Escalating concentrations of FXIII had a significant effect on cell survival at both 24 (solid bars) and 48 (hatched) hours after seeding. FXIII also had a dramatic inhibitory effect on cell proliferation measured over a 24 hour time period. This illustrated that the effect of FXIII was to keep the cells in statis and to prevent cell division in the capules. Cell division is undesirable in the encapsulated cells, as it will encourage the cells to break out of the capsule as an uncontrolled (premature) event.

### EXAMPLE 9

### Preparation of Transgene Encapsulated Cells

Briefly, 40 micrograms of Beta-Galactosidase plasmid was added to 200 microlitres of 4% agarose and empty capsules were prepared. Capsules were then stained with ethidium bromide and observed under UV light. Incorporation of the plasmid was observed, showing incorporation of the DNA in the agarose microcapsules.

The present inventors have found good transfection achieved by incorporating the gene or a plasmid containing the gene of interest into the capsule. As the capsule degrades, the cell uptakes the gene, likely by endocytosis.

### EXAMPLE 10

### Encapsulated Bone Marrow Stromal Cells

The present inventors investigated the viability of encapsulated rat bone marrow stromal cells (BMSC). The plasticity, pluripotentiality, ease of isolation, and high in vitro expansion potential make bone marrow stromal cells (BMSC) an attractive tool for cell and gene therapy. Such cells must home to the diseased tissue, properly engraft into the extracellular matrix, and once engrafted differentiate down appropriate cell lineages. Non-hematopoietic cells referred to as mesenchymal stem cells or bone marrow stromal cells, have been investigated for their multipotential character (Bianco et al. 2001, Theise et al. 2002, Cognet et al. 2000, Jiang et al. 2002 ,Krause et al. 2001). These cells have been found to be multipotential (have the ability to differentiate into various cell types) and serve as long-lasting precursors for bone marrow, bone, cartilage, and connective tissues (Cognet et al. 1999).

Encapsulated cell viability was assessed using a dual stain for apoptosis and necrosis. The viability of adherent BMSCs was compared with BMSCs grown on polyHEMA coated wells, BMSCs encapsulated in agarose, BMSCs encapsulated in agarose supplemented with Fibronectin, BMSCs encapsulated in agarose supplemented with Fibronectin and Fibrinogen.

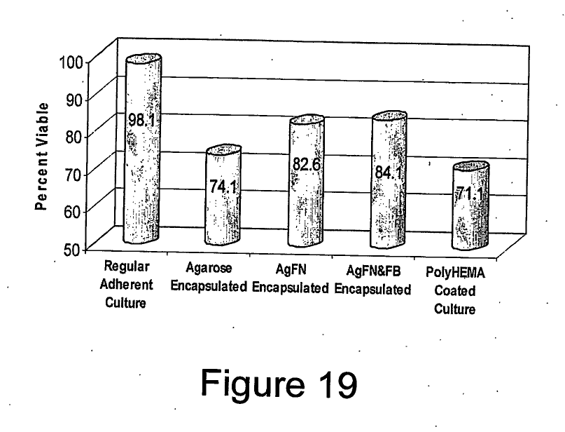

As shown in FIG. 19, the viability of the encapsulated BMSCs was increased significantly by the incorporation of Fibronectin (82.6%) and Fibronectin and Fibrinogen (84.1%).

### EXAMPLE 11

### Microencapsulation Enhances Engraftment of Bone Marrow Stromal Cells in Lung Microvasculature

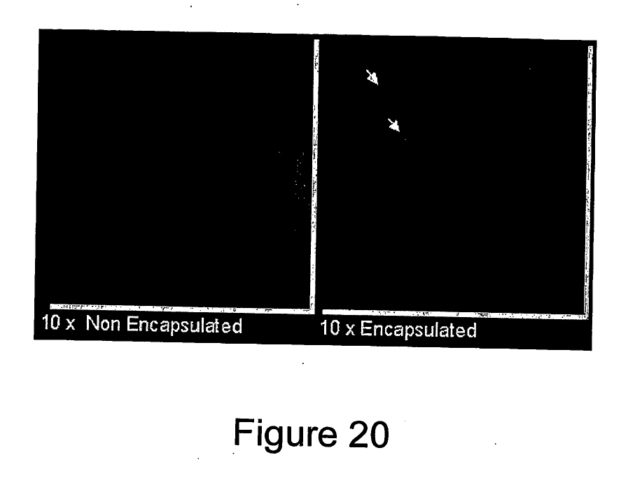

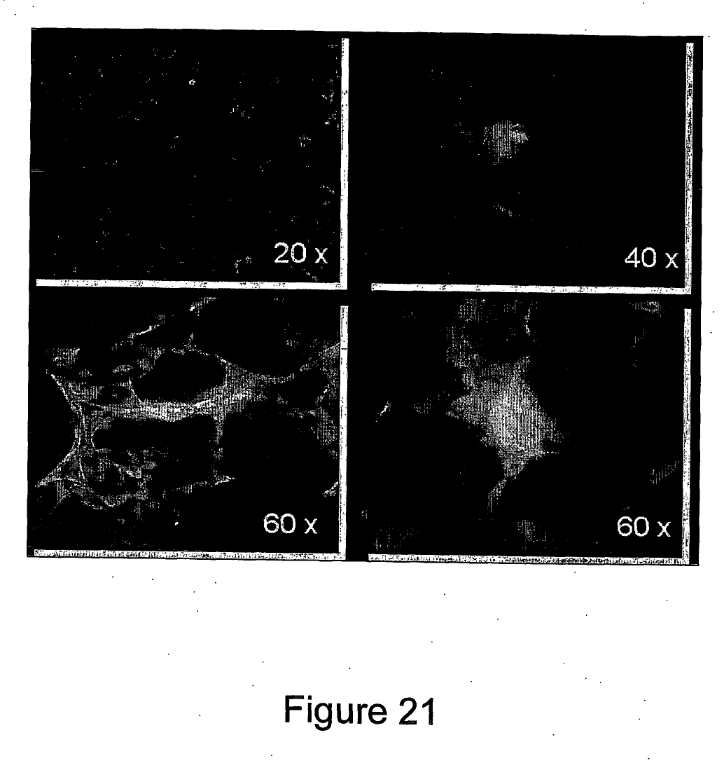

The present inventors investigated the role of encapsulation in improving the engraftment potential of bone marrow stromal cells in the lung microvasculature. CMTMR labeled cells were delivered to normal Fisher 344 rats by injection into the jugular vein. Animals were sacrificed at selected time points (15 minutes, 1d, 3d, & 7d) and the lungs were excised and examined. Tissues from the lung were fixed and analyzed by confocal microscopy. Results illustrate that there is significant engraftment of the non-encapsulated bone marrow stromal cells in the lung at 15 minutes. As shown in FIG. 20, there is no evidence of any CMTMR labeled non-encapsulated cells at 1 day and 3 days after injection. However, the encapsulation of the bone marrow stromal cells in enriched agarose resulted in engraftment of the fluorescently labeled cells in the lung microvasculature 3 days after delivery. The top panel of images in FIG. 21 illustrates the encapsulated cells one day after delivery at higher magnification. Encapsulated cells and clusters of cells were also observed at 7 days (lower panel) after delivery indicating long-term engraftment.

### EXAMPLE 12

### Viability of Encapsulated Human Fibroblasts

In the above examples, the present inventors examined the effect of encapsulation on rat fibroblast cells and rat bone marrow stromal cells. The present inventors also investigated the effect of encapsulation on human fibroblast cells.

Encapsulated cell viability was assessed using a dual stain for apoptosis and necrosis. The viability of non-encapsulated cells was compared with cells encapsulated in agarose and cells encapsulated in agarose supplemented with Fibronectin and Fibrinogen.

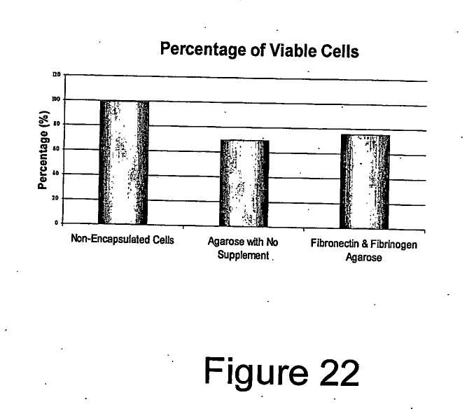

As shown in FIG. 22, the viability of the encapsulated human fibroblasts was significantly increased by the incorporation of Fibronectin and Fibrinogen into the agarose matrix,

It is to be understood that only the preferred embodiments have been shown, and that modifications thereof would be readily apparent to one skilled in the art. Therefore, the true scope and spirit of the invention resides in the appended claims and their legal equivalents, rather than by the given examples.

### REFERENCES

Bordignon, C. et al, Cell Therapy: Achievements and Perspectives (1999), Haematologica, 84, pp.1110-1149.

Lindvall, O. and Hagell, P., Cell Therapy and Transplantation in Parkinson's Disease (2001), Clinical Chemistry and Laboratory Medicine, 39, pp. 356-361.

Miyamoto, M, Current Progress and Perspectives in Cell Therapy for Diabetes Mellitus (2001), Human Cell, 14, pp. 293-300.

Morris, P. J., Immunoprotection of Therapeutic Cell Transplants by Encapsulation (1996), Trends in Biotechnology, 14, pp. 163-167,

Rowely, J. A. et al, Alginate Hydrogels as Synthetic Extracellular Matrix Materials (1999), Biomaterials, 20, pp. 45-53.

Ruoslahti, E., RGD and Other Recognition Sequences for Integrins (1996), Annual Review in Cell and Developmental Biology, 12, pp. 697-715.

Uludag et al., Technology of Mammalian Cell Encapsulation (2000), Advanced Drug Delivery Reviews, 42, pp. 29-64.

Bianco P, Riminucci M, Gronthos S, Robey P G. “Bone marrow stromal stem cells: nature, biology and potential applications”. Stem Cells (2001) 19: 180-192.

Brown M D, Schatzlein A G, Uchegbu I F. “Gene delivery with synthetic (non viral) carriers”. International journal of phannaceutics (2001) 229: 1-21.

Bally M B, Harvie P, Wong F, Kong S, Wasan E K, Reimer D L. “Bilogical barriers to cellular delivery of lipid-based DNA carriers.”. Advanced Drug Delivery Reviews (1999) 38: 291-315.

Campbell A, Kuliszewski M A., Stewart D J. “Cell-based gene transfer to the pulmonary vasculature: endothelial nitric oxide synthase overexpression inhibits monocrotaline-induced pulmonary hypertension” American Journal of Respiratory Cell and Mol. Biol. (1999) 21:567-575.

Colter D C, Sekiya I, Prockop D J. “Identification of a subpopulation of rapidly self-renewing and multipotential adult stem cells in colonies of human marrow stromal cells”. PNAS (2001) 98: 7841-7845.

Cognet P, Minguell J. “Adenoviral-mediated gene transfer into ex vivo expanded human bone marrow mesenchymal progenitor cells”. Experimental Hematology (2000) 28: 382-390.

Conget P A., Minguell J. “Phenotypical and Functional Properties of Human Bone Marrow Mesenchymal Progenitor Cells”. Journal of Cellular Physiology (1999) 181:67-73.

Ding L, Lu S, Batch R B, Sailors R L, Mushy N C. “Bone marrow stromal cells as a vehicle for gene transfer” Gene Therapy (1999) 6:1611-1616.

Eliceiri B P, Cheresh D A. “The role of alpha v integrins during angiogenesis: insights into potential mechanisms of action and clinical development”. The Journal of Clinical Investigation (1999) 103: 1227-1230.

Fukai F, Mashimmo M, Akiyama K, Goto T, Tanuma S, Katayama T. “Mudulation of apoptotic cell death by extracellular matrix proteins and a fibronectin-derived antiadhesive peptide”. Experimental Cell Research (1998) 242: 92-99.

Gailit J, Clarke C, Newman D, Tonnesen M G, Mosessen M W, Clark R A F. “Human fibroblasts bind directly to fibrinogen at RGD sites through integrin alpha V beta 3”. Experimental Cell Research (1997) 232: 118-126.

Giaid A, Yanagisawa M, Langleben D, Michel R P, Levy R, Shennib H, Kimura S, Masaki T, Duguid W P., Stewart D J. “Expression of endothelin-1 in the lungs of patients with pulmonary hypertension” The New England Journal of Medicine (1993) 328:1732-9.

Gelband C H., Katovich M J, Raizada M K. “Current perspectives on the use of gene therapy for hypertension” Circulation Research (2000) 87:1118-1122.

Giaid A, Saleh D. uReduced expression of endothelial nitric oxide synthase in the lungs of patients with pulmonary hypertension” N. Eng. J. of Med. (1995) 333:214-221.

Giancotti F G. “Integrin signaling: specificity and control of cell survival and cell cycle, progression”. Current Opinion in Cell Biology (1997) 9: 691-700.

Gomez-Navarro J, Contreras J L, Arafat W, Jiang X L, Krisky D, Oligino T, Marconi P, Hubbard B, Glorioso J C, Curiel D T, Thomas J M. “Genetically modified CD34+ cells as cellular vehicles for gene delivery into areas of angiogenesis in a rhesus model”. Gene Therapy (2000) 7:43-52.

Gravanis M B. Cardiovascular Pathophysiology. McGraw Hill Book Company. New York: 1987.

Gronthos S, Simmons P J, Graves S E, Robey P G. “Integrin-mediated interactions between human bone marrow strmal precurson cells and the extracellular matrix”. Bone (2001) 28: 174-181.

Jiang Y, Jahagirdar B M, Reinhardt R L, Schwartz R E, Keene C D, Ortiz-Gonzalez X R, Reyes M, Lenvik T, Lund T, Blackstad M, Du J, Aldrich S, Lisberg A, Low W, Largaespada D A, Verfaillie C M. “Pluripotency of senchymal stem cells derived from adults marrow”. Nature advance online publication (2002) 1-9.

Krause D S, Thiese N D, Collector M I, Henegariu O, Hwang S, Gardner R, Neutzel S, Sharkis S J. “Multi-organ, multi-lineage engraftment by a single bone marrow-derived stem cell”. Cell (2001) 105:369-377.

Krause D S. “Plasticity of marrow-derived stem cells”. Gene Therapy (2002) 9: 754-758.

Kamiya H., Tsuchiya H., Yamazaki J., Harashima H. “Intracellular trafficking and transgene expression of viral and non-viral gene vectors.”. Advanced Drug Delivery Reviews. 2001. 52:153-164.

Kotton D N, Ma B Y, Cardonso W V, Sanderson E A, Summer R S, Williams M C, Fine A. “Bone marrow-derived cells as progenitors of lung alveolar epithelium”. Development (2001) 128: 5181-5188.

Minguel J J., Erices A, Cognet P. “Mesenchymal Stem Cells”. Exp. Biol. Med. (2001) 226: 507-520.

Pasqualini R, Bodorova J, Ye S, Hemler M. “A study of the structure, function and distribution of beta 5 integrins using novel anti beta 5 monoclonal antibodies”. Journal of Cell Science (1993) 105: 101-111.

Pereira R F, Halford K W, O'Hara M D, Leeper D B, Sokolov B P, Pollard M D, Bagasra O, Prockop D J. “Cultured adherent cells from marrow can serve as long-lasting precursor cells for bone, cartilage, and lung in irradiated mice”. Proc. Natl. Acad. Sci. USA. (1995) 92:4857-4861.

Plath T, Detjen K, Weizel M, Marschall Z, Murphy D, Schirner M, Wiedenmann B, Rosewicz S. “A novel function for the tumor suppressor p16INK4a. Induction of anoikis via upregulaation of the alpha 5 beta 1 fibronectin receptor”. The Journal of Cell Biology. (2000) 150:1467-1477.

Powell K T, Weaver J C. Gel microdroplets and flow cytometry: rapid determination of antibody secretion by individual cells within a cell population. Biotechnology (1990) 4: 333-7.

Reinecke H, Murry C E. “Taking the death toll after cardiomyocyte grafting: a reminder of the importance of quantitative biology”. J Mol Cell Cardiol (2002) 34: 251-253.

Reinecke H, Zhang M, Bartosek T, Murry C E. “Survival, Integration, & Differentiation of Cardiomyocyte Grafts”. Circulation (1999) 100: 193-202.

Rubanyi, G. M. “The future of human gene therapy.”. Molecular Aspects of Medicine (2001) 22:113-142.

Scott G, Cassidy L, Busacco A. “Fibronectin suppresses apoptosis in normal human melanocytes through an inte grin-dependent mechanism”. The Journal of Investigative Dermatology (1997) 108: 147-153.

Stupack D G, Puente X S, Boutsaboualoy S, Storgard C M, Cheresh D A. “Apoptosis of adherent cells by recruitment of caspase-8 to unligated integrins”. The Journal of Cell Biology (2001) 155: 459-470.

Theise N D, Krause D S. “Toward a new paradigm of cell plasticity”. Leukemia (2002) 16: 542-548.

Thierry B, Molano D R., Pileggi A, Cattan P, Li H, Ricorde C, Inverardi L. “Patterns of engraftment in different strains of immunodificient mice reconstituted with human peripheral blood lymphocytes”. Transplantation (2001) 72:133-140.

Thurmond F A, Cronin E M, Willams S R. “A deadly game of musical chairs: survival of cells transplanted for myocardial repair”. J Mol Cell Cardiol. (2001) 33: 883-885.

Turcanu V. and Williams N. “Cell identification and isolation on the basis of cyokine secretion: A novel tool for investigating immune responses.”. Nature Medicine (2001) 7: 373-376.

Wang E, Marcotte R, Petroulakis E. “Signalling pathway for apoptosis: a racetrack for life and death.”. Journal of Cellular Biochemistry Supplements (1999) 32: 95-102.

Weaver J C, Bliss J G, Powell K T, Harrison G T, Williams G B. “Rapid clonal growth measurements at the single-cell level: gel microdroplets and flow cytometry”. Biotechnology (1991) 9: 873-7.

Weaver J C, McGrath P, Adams S, “Gel microdrop technology for rapid isolation of rare and high producer cells”. Nature Medicine (1997) 5: 583-5.

Young J L and Dean D A. “Nonviral gene transfer strategies for the vasculature”. Microcirculation (2002) 9: 35-50.

Zhang W W. “Development and application of adenoviral vectors for gene therapy of cancer”. Cancer Gene Therapy (1999) 6:113-38.

Zhang M, Methot D, Poppa V, Fuio Y, Walsh K, Murry C E. “Cardiomyocyte grafting for cardiac repair: graft cell death and anti-death strategies”. Joumal of Molecular Cell Cardiology (2001) 33: 907-921.

Zhang Z, Vuori K, Reed J C, Rouslahti E. “The alpha 5 beta 1 integrin supports survival of cells on fibronectin an up-regulates Bcl-2 expression”. PNAS (1995) 92: 6161-6165.

1-37. (canceled)

38. A method of preparing a prepared cell, comprising encapsulating said cell in a cell encapsulation medium in vitro to form an encapsulation product for use in cell therapy in vivo, wherein said encapsulation product includes an integrin binding partner.

39. The method as claimed in claim 38, wherein said integrin binding partner is selected from the group consisting of collagen, Fibronectin, Fibrinogen, laminin, thrombospondin, vitronectin, factor X, C3bi, lg-like cell adhesion molecule (ICAM-1,2,3), type 1 collagen, vascular cell adhesion molecule (VCAM-1), mucosal addressin cell adhesion molecule-1 (MAdCAM-1), vitronectin, collagens, laminin, LFA, Mac-1, tenascin, von Willebrand factor, thrombospondin, factor X, FXll, FXllla, Arg-Gly-Asp, Leu-Asp-Val, His-His-Leu-Gly-Gly-Ala-Lys-Gln-Ala-Gly-Asp-Val, an integrin binding partner containing the sequence Arg-Gly-Asp, Leu-Asp-Val, and an integrin binding partner containing the sequence His-His-Leu-Gly-Gly-Ala-Lys-Gln-Ala-Gly-Asp-Val.

40. The method as claimed in claim 39, wherein said integrin binding partner is Fibrinogen.

41. The method as claimed in claim 39, wherein said integrin binding partner is Fibronectin.

42. The method as claimed in claim 41, wherein said encapsulation product is FXIII.

43. The method as claimed in claim 39, wherein said encapsulation product is FXIIIa.

44. The method as claimed in claim 39, wherein said integrin binding partner contains the recognition sequence argine-glycine-asparate (RGD).

45. The method as claimed in claim 38, wherein said integrin binding partner is bound to said prepared cell.

46. The method as claimed in claim 45, wherein said integrin binding partner is bound to said prepared cell prior to encapsulation.

47. The method as claimed in claim 38, wherein said integrin binding partner is not bound to said prepared cell.

48. The method as claimed in claim 38, wherein said integrin binding partner is in said cell encapsulation medium.

49. The method as claimed in claim 38, wherein said integrin binding partner is at the surface of said cell encapsulation medium.

50. The method as claimed in claim 38, wherein said cell encapsulation medium is selected from the group consisting of agarose with fibrin, agarose with Fibronectin, a combination of Fibronectin and Fibrinogen, plant-derived gums, alkali metal alginates and agarose, cellulose and its derivatives, gelatin, chitosan and extracellular matrix (ECM) components.

51. The method as claimed in claim 38, wherein said cell encapsulation medium is a natural polymer compatible with the survival and function of said cell.

52. The method as claimed in claim 38, wherein said cell encapsulation medium is a synthetic polymer compatible with the survival and function of said cell.

53. The method as claimed in claim 38, wherein most of said encapsulation product comprises one prepared cell per encapsulation.

54. A method of preparing a prepared cell for use in vivo, comprising encapsulating said cell in a cell encapsulation medium in vitro to form an encapsulation product, wherein said encapsulation product includes an integrin binding partner, and wherein said encapsulation product contains one cell.

55. A method of preparing a prepared cell for storage or transportation for later use in vivo, comprising encapsulating said cell in a cell encapsulation medium in vitro to form an encapsulation product, wherein said encapsulation product includes an integrin binding partner.

56. The method as claimed in claim 38, wherein said cell encapsulation medium contains a transgene.

57. The method as claimed in claim 38, wherein said prepared cell contains a transgene.

58. The method as claimed in claim 57, wherein said transgene is incorporated into the cell subsequent to including a transgene in said encapsulation medium.

59. A method of providing cell therapy to a patient in need thereof, comprising administering the prepared cell of claim 38.

60. The method as claimed in claim 59, wherein said administration is intercardiac.

61. The method as claimed in claim 38, wherein said encapsulation product further comprises an external factor which can affect a host cell which is external to the encapsulation product.

62. The method as claimed in claim 61, wherein said external factor is selected from the group consisting of DCAM, ICAM and VCAM.

63. The method as claimed in claim 38, wherein said cell is selected from the group consisting of fibroblasts, endothelial cells, smooth muscle cells, progenitor/stem cells (e.g. from bone marrow, adipose, or peripheral blood), dermal fibroblasts, EPC (endothelial cells) or other mesenchymal cells, marrow stromal cells (MSC), and epithelial cells.

64. The method as claimed in claim 59, wherein said cell is selected from the group consisting of fibroblasts, endothelial cells, smooth muscle cells, progenitor/stem cells (e.g. from bone marrow, adipose, or peripheral blood), dermal fibroblasts, EPC (endothelial cells) or other mesenchymal cells, marrow stromal cells (MSC), and epithelial cells.

65. The method as claimed in claim 38, wherein said cell is selected from the group consisting of fibroblasts and bone marrow stromal cells.

66. The method as claimed in claim 59, wherein said cell is selected from the group consisting of fibroblasts and bone marrow stromal cells.

67. A kit for cell based therapy in a mammal, comprising an effective amount of an integrin binding partner and instructions for the use thereof to prepare a cell encapsulation medium.

68. The kit as claimed in claim 67, wherein said instructions further describe administration of the cell based therapy to a patient in need thereof.

69. The kit as claimed in claim 68, wherein said instructions describe administration by cell based gene therapy.

70. The kit as claimed in claim 67, further comprising an encapsulation medium.

71. The kit as claimed in claim 69, wherein said instructions describe administration using viable, transfected mammalian cells, said transfected mammalian cells containing at least one expressible trans-gene coding for an apoptosis inhibitor.

72. The kit as claimed in claim 71, wherein said mammalian cells are selected from the group consisting of dermal fibroblasts, smooth muscle cells, progenitor cells, endothelial progenitor cells, epithelial progenitor cells, smooth muscle progenitor cells, stem cells, and endothelial cells.

73. The kit as claimed in claim 67, wherein said integrin binding partner is selected from the group consisting of collagen, Fibronectin, Fibrinogen, laminin, thrombospondin, vitronectin, factor X, C3bi, lg-like cell adhesion molecule (ICAM-1,2,3), type 1 collagen, vascular cell adhesion molecule (VCAM-1), mucosal addressin cell adhesion molecule-1 (MAdCAM-1), vitronectin, collagens, laminin, LFA, Mac-1, tenascin, von Willebrand factor, thrombospondin, factor X, FXlIl, FXllla, Arg-Gly-Asp, Leu-Asp-Val, His-His-Leu-Gly-Gly-Ala-Lys-Gln-Ala-Gly-Asp-Val, an integrin binding partner containing the sequence Arg-Gly-Asp, Leu-Asp-Val, and an integrin binding partner containing the sequence His-His-Leu-Gly-Gly-Ala-Lys-Gln-Ala-Gly-Asp-Val.

74. The kit according to claim 67, wherein said cell encapsulation medium is selected from the group consisting of agarose with fibrin, agrarose with Fibronectin, a combination of Fibronectin and Fibrinogen, plant-derived gums, alkali metal alginates and agarose, cellulose and its derivatives, gelatin, chitosan and extracellular matrix (ECM) components.


SEQUENCE LISTING (3 sequences)

SEQ ID NO: 1 - protein, 3 aa, Artificial
  binding motif
RGD

SEQ ID NO: 2 - protein, 3 aa, Artificial
  binding motif
LDV

SEQ ID NO: 3 - protein, 12 aa, Artificial
  binding motif
HHLGGAKQAGDV